(19) United States
(12) Patent Application Publication
(10) Pub. No.: US 2005/0009007 A1
(43) Pub. Date: Jan. 13, 2005

(54) Methods and systems for monitoring cell exocytosis or endocytosis

(76) Inventors: Leon Lagnado, Cambridge (GB); Artur Llobet, Cambridge (GB)

Correspondence Address: Intellectual Property Department;DEWITT ROSS & STEVENS S.C., US Bank Building, 8000 Excelsior Drive Suite 401, Madison, WI, 53717-1914, US

(21) Appl. No.: 10/881,063

(22) Filed: Jun. 30, 2004

Related U.S. Application Data
(60) Provisional application No. 60/485,538, filed on Jul. 8, 2003.

(51) Int. Cl. C12Q 1/00

(52) U.S. Cl. 435/4

(57) ABSTRACT

A method of assaying exocytosis or endocytosis, comprises: providing a transparent substrate with at least one cell membrane mounted thereon apposing the substrate, provoking a change in the amount of exocytosis or endocytosis at the membrane, obtaining light signals from the region of the substrate which the membrane apposes using interference reflection microscopy, and analysing the light signals to detect the exocytosis or endocytosis at the membrane.

### CROSS-REFERENCE TO RELATED APPLICATIONS

This application claims priority under 35 USC §119(e) to U.S. Provisional Patent Application 60/485,538 filed 8 Jul. 2003, the entirety of which is incorporated by reference herein.

### FIELD OF THE INVENTION

The present invention relates to methods and systems for monitoring cell exocytosis or endocytosis using interference reflection microscopy (IRM).

### BACKGROUND OF THE INVENTION

The traffic of secretory vesicles to the plasma membrane of eukayrotic cells is essential for normal cellular function. It also forms the basis of intercellular communication in multicellular organisms through the release of a wide array of extracellularly acting molecules. The process of secretion is generally classified into two types: regulated and constitutive (Burgoyne and Morgan, 2002). Regulated secretion takes place when a vesicle or granule rapidly fuses with the surface membrane (exocytosis) in response to an external stimulus, releasing its contents. In contrast constitutive secretion is less tightly coupled to a stimulus. Constitutive secretion is involved in the release of extracellular molecules (e.g plasma proteins, antibodies, extracellular matrix components) as well as the insertion of proteins into the surface membrane. However, it is now known that constitutive exocytosis and endocytosis can also be regulated by stimuli such as hormones and neurotransmitters (Royle and Murrell-Lagnado, 2003).

Regulated exocytosis has been extensively studied in neural synapses where synaptic vesicles (diameter 30-50 nm) fuse with the plasma membrane in response to a rise in calcium concentration inside the cell, thereby releasing the neurotransmitter substances they contain. Certain neurons also possess granules (also known as large dense-core vesicles) that can be triggered to undergo exocytosis independently of synaptic vesicles (Rettig and Neher, 2002). Granules have diameters of about 160-350 nm (Parson et al., 1995). A wide range of non-neuronal cell types also contain regulated secretory vesicles or secretory granules, the contents of which serve a diverse range of physiological functions. These include cells specialized to secrete a wide variety of secretory products including, for example, neuroendocrine, endocrine, exocrine, paracrine and haemopoietic cells, as well as cells where exocytosis serves a more specialized and transitory function, such as the sperm and the egg. The widespread and fundamental importance of regulated exocytosis has been reviewed by Burgoyne and Morgan (2003). The maintenance of normal secretory function requires that exocytosis be followed by endocytosis to retrieve excess membrane and associated proteins. Thus, endocytosis is coupled to both regulated and constitutive exocytosis (Gundelfinger et al., 2003; Royle and Murrell-Lagnado, 2003).

The importance of secretory processes has stimulated the development of a number of methods for assaying exocytosis and endocytosis in living cells in real-time (Betz et al., 1996; Angleson and Betz, 1997; Burgoyne and Morgan, 2003). Methods used to detect secretion can be classified into two categories: analytical (or biochemical) and real-time. Analytical techniques detect the substance released to the extracellular medium by the cells via a secretory process. Two widely used techniques for detecting such substances are ELISA (enzyme-linked immunosorbent assay) and radioimmunoassay (RIA). Analytical methods rely on detecting the substance released, rather than the process of secretion itself, and are therefore slow and specific to the chemical being detected. They are not easily applied to single cells and are costly in the use of reagents.

Real-time methods for measuring secretion include, measurement of membrane capacitance, amperometry and use of fluorescent dyes or proteins (Angelson and Betz, 1997; Ryan, 2001). One of the most direct approaches is the capacitance technique, which measures changes in membrane surface area associated with fusion and retrieval of vesicles (Neher and Marty, 1982). Capacitance measurements depend on voltage-clamping a cell behaving as a single electrical compartment (Gillis, 1995), confining studies of exocytosis and endocytosis at the synapse to a few neurons with giant terminals, such as bipolar cells from the goldfish retina (von Gersdorff and Matthews, 1994), frog hair cells (Parsons et al., 1994) and the Calyx of Held in slices of rat brain-stem (Sun and Wu, 2001). A second drawback of the capacitance technique is that it cannot monitor exocytosis during normal electrical activity because voltage-sensitive conductances, such as sodium and calcium channels, can generate spurious signals (Horrigan and Bookman, 1994; Gillis, 1995). Further, the capacitance technique is invasive; the properties of exocytosis and endocytosis can be severly altered by the dialysis of the cell interior through the whole-cell pipette. Nonethless, capacitance measurements have been used to study exocytosis and endocytosis in a number of cell types, including mast cells (Riedel et al., 2002), neutrophils (Lollike and Lindau, 1999), retinal photoreceptors (Rieke and Schwartz, 1994), dorsal root ganglion neurons (Huang and Neher, 1996), pancreatic alpaa and beta cells (Barg, 2003), gonadotrophs (Thomas and Waring, 1997), chromaffin cells (Neher and Marty, 1982), melanotrophs (Sikdar et al., 1998), human growth hormone-secreting adenoma cells (Takei et al., 1998), macrophages (Holevinsky and Nelson, 1998), basophils (Oshiro et al., 1997), airway secretory epithelia (Chen et al., 2001) and endothelial cells (Carter et al., 1998).

Amperometry is a method that detects the secretion of chemicals that can be oxidized (reviewed by Michael and Wightman, 1999). The oxidation occurs on the surface of an electrode placed near the cells. A major drawback of amperometry is that only some secreted substances can be detected.

Fluorescence imaging techniques for studying exocytosis and endocytosis generally involve labeling vesicles or granules with a membrane dye such as FM1-43 (Cochilla et al., 1999) or a GFP fusion-protein such as synapto-pHluorin (Ryan, 2001). These imaging methods generally have lower time-resolution than the capacitance technique. Toxic photodamage induced by the intense lights required to excite fluorescence is an important drawback and limitation of these methods. Further, to detect the dim fluorescence emission very sensitive detectors or cameras are required.

In view of the above, alternative methods and systems are needed that can reliably detect exocytosis and endocytosis in real-time and overcome some or all of the above drawbacks

### SUMMARY OF THE INVENTION

We provide an optical approach for monitoring exocytosis and endocytosis. The approach, which can operate in real-time, uses interference reflection microscopy (IRM) to visualize areas of destructive interference occurring when light is reflected from a region of cell membrane in close apposition (typically <100 nm) to a transparent substrate such as a glass coverslip (Curtis, 1964; Gingell and Todd, 1979; Weber, 2003).

IRM has been used to study cell adherence and motility (Niewohner et al., 1997; Anderson and Cross, 2000). It has also been used to image spontaneous discharge of water from contractile vacuoles in Dictyostelium amoebae (Heuser et al., 1993; Betz et al., 1996). However, we now demonstrate ways in which IRM can be used to study the stimulation or inhibition of exocytosis and endocytosis. We also show how IRM can be used to monitor exocytosis and endocytosis mediated by small-sized (e.g. less than 500 [symbol] m diameter) cell organelles.

For example, at synapses stimulated to release neurotransmitter from small vesicles, IRM can be used to monitor the resultant expansion of the surface membrane as an increase in the area of close contact between the membrane and substrate (the “footprint”). After vesicle fusion, retraction and removal of surface membrane by endocytosis can be monitored as a decrease in the area of the footprint. We show that IRM measurements of exocytosis and endocytosis at the giant terminal of goldfish bipolar cells are almost identical to those obtained using the capacitance technique or by use of FM1-43. On the other hand, distinctly different signals are obtained from chromaffin cells releasing the contents of dense-core secretory granules: in this case IRM visualizes fusion of individual granules as the appearance of discrete bright spots within the footprint. We show that the IRM resolution of fusion of single granules in space and time is comparable to the results obtained with total internal reflection fluorescence microscopy (TIRFM; Steyer et al., 1997; Duncan et al., 2003).

Application of IRM to the study of stimulated or inhibited secretion, or to the study of small organelles, provides a new approach that can be relatively cheap and easy to implement but can also be used in conjunction with electrophysiological techniques, such as capacitance measurements, and fluorescence imaging techniques, such as confocal microscopy and TIRFM. IRM can overcome two major drawbacks of the capacitance technique. First, exocytosis and endocytosis can be investigated while voltage-sensitive conductances are active because the cell does not need to be voltage-clamped. We have taken advantage of this to measure exocytosis in bipolar cells stimulated by current injection and demonstrate that exocytosis during normal electrical activity occurs at rates at least 10-fold slower than the maximum. Second, measurements can be made from neurons with small synaptic terminals and complex morphologies. We have taken advantage of this to investigate exocytosis and endocytosis in the synaptic terminal of bipolar cells from the mouse retina. The visualization of fusion by dense-core granules may also make IRM a useful method for studying Ca²⁺-triggered secretion in cell-free systems (Avery et al., 2000; Holroyd et al., 2002).

Further advantages of the IRM approach are that it is applicable to a wide-range of cell types and is independent of the substance(s) being secreted. It is also non-invasive, does not induce photodamage, and does not require a skilled operator.

In a first aspect, the present invention provides a method of assaying exocytosis or endocytosis, comprising:
providing a transparent substrate with at least one cell membrane mounted thereon apposing the substrate,
provoking a change in the amount of exocytosis or endocytosis at the membrane,
obtaining light signals from the region of the substrate which the membrane apposes using interference reflection microscopy, and
analysing the light signals to detect the exocytosis or endocytosis at the membrane.

The change in the amount of exocytosis or endocytosis can be provoked by stimulating exocytosis or endocytosis at the membrane, or by inhibiting exocytosis or endocytosis at the membrane.

Preferably the analysing step comprises quantifying the amount of exocytosis or endocytosis occurring at the membrane.

Typically, the membrane is a part of a cell, i.e. a cell is mounted on the transparent substrate such that a portion of its surface membrane apposes the substrate. Exocytosis or endocytosis can then be stimulated or inhibited generally in the cell. However, the method can also be applied to a “cell-free” system, in which the membrane is isolated from its parent cell (e.g. by disrupting the parent cell), and exocytosis or endocytosis is stimulated or inhibited specifically at the membrane.

The obtained light signals are typically, but not always, in the form of images of the region of the substrate which the membrane apposes, i.e. is in close contact to.

However, the light signals may also measure the total amount of light reflected back from the region of the substrate which the membrane apposes.

The cell (or cell from which the cell membrane is isolated) may be a neural cell. Alternatively it may be a cell of the immune or endocrine system, or an endothelial cell, or a cell of haemopoietic lineage, or a macrophage. The cell may be acutely dissociated, or maintained in culture. The cell may be from a cell line. The cell may be mammalian or non-mammalian. The cell may be from a proliferated population, for instance of cultured mast cells or basophils.

Preferably a plurality of cells/cell membranes are mounted on the substrate. The light signals may then be obtained simultaneously from more than one cell/cell membrane.

The change in the amount of exocytosis or endocytosis may be due to more than one exocytic or endocytic event.

The exocytosis or endocytosis may be mediated by vesicles e.g. synaptic vesicles. Alternatively, the exocytosis or endocytosis may be mediated by secretory granules. Vesicles typically have diameters in the range 30-50 nm and granules typically have diameters in the range 150-350 nm. We have shown that the method may be used to detect exocytosis or endocytosis mediated by organelles that have diameters of less than about 500 nm.

Preferably, the analysing step comprises measuring the area of the membrane apposing the substrate.

Alternatively or additionally, the analysing step comprises identifying the individual cell organelles (e.g. granules or vesicles) which mediate exocytosis or endocytosis.

Any one or combination of the preferred/optional features of the first aspect of the invention are applicable to the following further aspects.

A further aspect of the present invention provides a method of monitoring exocytosis or endocytosis mediated by organelles of less 500 nm diameter, comprising:
providing a transparent substrate with at least one cell membrane mounted thereon apposing the substrate,
obtaining light signals from the region of the substrate which the membrane apposes using interference reflection microscopy, and
analysing the light signals to detect (and preferably to determine the amount of) exocytosis or endocytosis at the membrane mediated by said organelles.

This aspect of the invention may be used to monitor constitutive secretion mediated by small organelles. For the first time we show that IRM can be used to image individual such organelles.

The present invention also has particular utility as a screening assay. For example, the effect a bioactive agent may have on the exocytic or endocytic response of a cell can be tested by introducing that bioactive agent to the cell. The introduced bioactive agent may then directly stimulate or inhibit secretion, or the bioactive agent may have an effect on secretion stimulated or inhibited in other ways (e.g. electrically, or by changing the chemical environment of the cell). Whichever way the change in the amount of secretion is provoked, the secretion can be detected by analysis of the IRM light signals. Such analysis will generally be much quicker to complete than e.g. biochemical assays (which attempt to detect the chemical that is released). The speed at which the signals can be obtained and analysed makes high throughput screening assays based on the method highly attractive. Also, in contrast, to biochemical assays, the method is not specific to particular secreted substances, but has more general applicability.

Particularly for screening applications, it is useful to compare the extent of exocytosis or endocytosis before and after provocation. Therefore, a further aspect of the invention provides a method of assaying exocytosis or endocytosis, comprising:
providing a transparent substrate with at least one cell membrane mounted thereon apposing the substrate,
obtaining first light signals from the region of the substrate which the membrane apposes using interference reflection microscopy
provoking a change in the amount of exocytosis or endocytosis at the membrane,
obtaining second light signals from said region of the substrate using interference reflection microscopy, and
analysing the first and second light signals to detect (and preferably quantify) the change in the amount of exocytosis or endocytosis at the membrane.

Thus, for example, exocytic and endocytic activity before contact with a bioactive agent can be compared with activity after, to assess whether the bioactive agent increases or decreases exocytic or endocytic activity.

In a further aspect, the present invention provides a method for screening a population of cells to identify cells with an altered phenotype with respect to exocytosis or endocytosis,
wherein the method comprises:
(a) for each cell of the population:
   providing a transparent substrate with the cell mounted thereon such that a portion of its surface membrane apposes the substrate,
   obtaining light signals from the region of the substrate which the membrane apposes using interference reflection microscopy, and
   analysing the light signals to determine the amount of exocytosis or endocytosis at the membrane; and
(b) comparing the amounts of exocytosis or endocytosis thus-determined to identify those cells of the population which have an altered phenotype with respect to exocytosis or endocytosis.

The method may further comprise provoking a change in the amount of exocytosis or endocytosis at the membrane of each cell before the step of obtaining light signals.

The cells may be from a proliferated population.

In a further aspect, the present invention provides an apparatus for monitoring, assaying or screening exocytosis or endocytosis comprising:
a transparent substrate on which at least one cell membrane can be mounted to appose the substrate,
an interference reflection microscope system configured to obtain light signals from the membrane, and
a computerised signal analyser operatively connected to the microscope system, the signal analyser being adapted to detect exocytosis or endocytosis at the membrane in the light signals.

The apparatus can be used in the performance of the method of any of the previous aspects of the invention.

An advantage of the apparatus is that the signal analyser automatically detects exocytosis or endocytosis, thereby making the apparatus particularly suitable for use in high throughput screening of bioactive agents or the screening of cell populations. Preferably the signal analyser is adapted to determine the amount of exocytosis or endocytosis at the membrane.

Typically, the substrate is incorporated into a multi-well plate, whereby each well of the plate effectively has a respective substrate.

To enhance system automation, the apparatus may further comprise a first transport device for moving such a multi-well plate relative to the microscope system such that light signals can be obtained from successive wells of the multi-well plate by the microscope system.

To further enhance system automation, the microscope system may utilize a plurality of objective lenses to allow simultaneous light collection for interference reflection microscopy from respective wells of a multi-well plate.

Additionally or alternatively, the apparatus may further comprise an electrical stimulating probe for electrically stimulating exocytosis or endocytosis in cells, and a second transport device for bringing the probe to successive wells of the multi-well plate. The first and second transport devices may be combined in a single transport device in an apparatus in which the multi-well plate is moved relative to the microscope system and the stimulating probe.

Furthermore, the apparatus may comprise systems for application and removal of fluids and substances from cells or cell membranes on the multi-well plate.

The apparatus may further comprise a photolytic illumination system. Such a system can be used to illuminate the plate with intense light and thereby photolytically release bioactive agents. So-called “caged compounds” can be released in this way, including caged calcium, peptides and proteins (Curley and Lawrence, 1999).

### BRIEF DESCRIPTION OF THE DRAWINGS

The invention will be described by way of example with reference to the accompanying drawings.

FIG. 1. Interference Reflection Microscopy (IRM) at the giant synaptic terminal of bipolar cells from the goldfish retina
A. Schematic diagram showing the basic principle of IRM.
B. Schematic diagram showing an implementation of IRM. Note that we did not use the antiflex method (Ploem et al., 1975) to enhance contrast.
C. (a) Transmitted light image of the giant synaptic terminal of a depolarizing bipolar cell isolated from the retina of goldfish and (b) corresponding IRM image showing the “footprint” where membrane is in close apposition to glass.
D. Difference images showing (a) the footprint in C at rest, (b) 0.5 s after the start of depolarization and (c) 15.5 s after the end of the stimulus. The outlines of the footprints are shown in (d), the bold outline showing the outline of the expanded footprint in (b). The stimulus was a depolarization of 500 ms.
E. The time-course of the change in area of the footprint in D (as a percentage of the area at rest). The timing of the depolarization is shown by the solid bar.

FIG. 2. Changes in the area of the footprint reflected Ca²⁺-triggered exocytosis followed by endocytosis
A. Averaged responses to a 500 ms depolarization recorded with patch pipettes containing 0.4 mM BAPTA (thick trace, n=19), 500 nM botulinum toxin E light chain (thin trace, n=4) and 10 mM BAPTA (thin broken trace, n=7). The averaged Ca²⁺ currents are shown below.
B. Comparison of the averaged time-course of endocytosis measured by IRM (thick trace) and capacitance (thin trace) in the same group of 8 cells. The stimulus was a 0.5 s depolarization (bar).

FIG. 3. Changes in the area of the footprint closely reflected the kinetics of exocytosis and endocytosis
A. Single response triggered by a 20 ms depolarization (at arrow). The recovery phase followed a single exponential with a time-constant of 1.1 s, as shown by the smooth line.
B. Single response triggered by a 5 s depolarization (black bar). Three phases of exocytosis were observed. First, release of the RRP increased the area by 1.8% (dashed arrow). Second, the area increased at a rate of 1.47% s⁻¹ (first thick straight line) for 2.5 s. The third phase occurred at 0.22% s⁻¹ (second thick straight line). The area continued to increase for ^{˜}3 s after the end of the stimulus before recovering back to baseline.
C. Average response triggered by a 5 s depolarization (thick trace, n=10). The averaged FM1-43 signal generated by the same stimulus in another group of cells is shown by the thin trace (n=19, from Neves et al., 2001).

FIG. 4. Exocytosis and endocytosis measured during electrical activity
A. Recordings of membrane voltage (Vm) and changes in the area of the footprint in a bipolar cell under current-clamp. Current injections of 2 pA and 20 pA were applied for 5 s (bars).
B. Comparison of the increase in membrane area measured under current-clamp (2 pA current; thin trace, n=4) and voltage-clamp (depolarization to −10 mV; thick trace, n=4). The slope of the lines describing the second phase of exocytosis were 0.39% s⁻¹ (thick straight line) and 3.9% s⁻¹ (thin straight line).

FIG. 5. Exocytosis and endocytosis measured in the synaptic terminal of a mouse neuron
A. Transmitted light image of depolarizing bipolar cell isolated from the retina of a mouse.
B. IRM image showing (a) the “footprint” of a mouse bipolar cell, and the difference images (b) at rest, (c) 1.52 s after the beginning of a 0.5 s depolarization and (d) 19 s after the end of the stimulus.
C. The time-course of the change in area of the footprint in B. (as a percentage of the area at rest). The timing of the depolarization is shown by the solid bar.
D. Averaged response triggered by a 0.5 s depolarization (n=17 cells). Two phases of exocytosis were observed. First, release of the RRP increased the area by 0.5%. Second, the area increased at a rate of 3.2% s⁻¹ (thick straight line).

FIG. 6. Fusion of individual granules in bovine chromaffin cells visualized by IRM and TIRFM
A. (a) Footprint of a chromaffin cell, and difference images showing the footprint (b) at rest, (c) 0.36 s after the beginning of a 0.5 s depolarization and (d) 3 s after the end of the stimulus.
B. The time-courses of the intensity changes at the centre of the bright spots arrowed in A.
C. Visualization of the fusion of a single granule by simultaneous IRM (left) and TIRFM (middle). The right-hand side shows the superimposition of the TIRFM image and IRM images. Release of FM4-64 colocalised with the appearance of a bright-spot in the footprint.
D. Comparison of the averaged time-course of the IRM and FM4-64 signals at the center of 11 different fusion events from 6 cells. The IRM signal is shown by the thick trace and the FM4-64 signal is shown by the thin trace. Note that the release of FM4-64 occurred in a burst that declined rapidly, while the IRM signal rose more slowly and did not recover on this time-scale.

FIG. 7. Fusion of granules visualized by IRM correlated with the capacitance response
A. The relation between the capacitance increase in a chromaffin cell and the number of granules fusing per unit area of the footprint. Each point represents a simultaneous measurement from one cell (n=19 cells). The correlation coefficient was 0.72.
B. The relation between the capacitance increase in a chromaffin cell and the total number of granules fusing, estimated as described in the text. The line through the points has a slope of 1.43 fF/granule.
C. The relation between the capacitance increase in a chromaffin cell, expressed as a percentage of the resting capacitance, and the change in the area of the footprint. Each point represents simultaneous measurements from one cell, made immediately after the depolarizing stimulus. The change in the area of the footprint did not correlate with the increase in surface area measured by capacitance.

FIG. 8. The basis of the different IRM signals in synaptic terminals and chromaffin cells

Schematic diagrams showing (left) the rapid and full collapse of a synaptic vesicle, resulting in membrane expansion and enlargement of the footprint detected by IRM, and in contrast (right) fusion of a dense-core granule in a chromaffin cell generating a spatially distinct invagination of the surface membrane that is detectable as the appearance of a bright spot within the footprint.

### DETAILED DESCRIPTION

Experimental Procedures

Interference Reflection Microscopy

The theory of image formation has been described in detail by Izzard and Lochner (1976) and Verschueren (1985). The method depends on the fact that light is reflected at the interface between media of different refractive indices. When a cell on a coverslip is observed under epi-illumination using an oil-immersion objective, the first two interfaces where the light is reflected are the glass-solution and the solution-cell boundaries (FIG. 1A). If the thickness of the aqueous solution is of the order of the wavelength of light used (λ), the two reflected beams, R₁ and R₂, interfere. The optical path difference (Δ) between R₁ and R₂ depends on the distance between the interfaces (d). If d=0, Δ=λ/2, and there is maximum destructive interference. The two beams are half a wavelength out of phase because R₁ is reflected at the interface between a medium of high refractive index (glass, n₁) and lower refractive index (aqueous solution, n₂), causing a phase change of −π. There is no phase change for R₂ because it is reflected at the interface between the aqueous solution and cell membrane of higher refractive index (n₃). The IRM image of the region around a cell is bright because only the reflected beam R₁ is imaged. The dark “footprint” occurs in the region where the membrane is separated from the glass by distances less than about 100 nm, causing destructive interference between reflected beams R₁ and R₂ (FIG. 1C). Increased separation between membrane and glass in this region causes the footprint to brighten. In general, maximal destructive interference occurs when Δ=Nλ, and constructive interference occurs when Δ=(N+½)λ is an integer. Thus interference patterns can also arise due to reflections at structures within the cell or at its upper surface. These contributions can be minimized by using objectives of high numerical aperture, allowing the selective visualization of the zero-order interference pattern arising from optical path differences less than one wavelength (Izzard and Lochner, 1976).

We implemented IRM in a simple way, shown in FIG. 1B. An inverted epi-fluorescence microscope (Zeiss Axiovert 135 M) was used with a half-silvered mirror (Omega Optical) in the filter block. Illumination was provided by white light from a tungsten filament lamp of 150 W and imaging performed with a 63× objective with numerical aperture of 1.4. The image was magnified two-fold with a telescope placed between the microscope and the CCD camera (Cohu 4910 series). The contrast and gain were altered using an Argus 10 image processor (Hamamatsu) and the video signals were digitized by an LG-3 board (Scion Corporation) driven by NIH Image software. Image acquisition was triggered by a TIL signal from a second computer used for electrophysiology. Monochromatic light is usually used for IRM (Izzard and Lochner, 1976), but we found that the signal-to-noise ratio in measurements of the area of the footprint improved with higher intensities and therefore used white light. The contrast in IRM images can be degraded by scattered light in the objective and this problem is often overcome using an antiflex objective with a rotatable quarter-wave plate on the front lens together with plane polarizers in the incident and reflected light paths (Ploem, 1975; Verschueren, 1985). We found that contrast enhancement with an image processor provided an easier solution for the applications described in this paper.

Analysis of IRM Images

Bipolar cell terminals produced relatively uniform footprints (FIG. 1C, FIG. 5B and FIG. 6A). The area of these footprints was measured in NIH Image or Image-J software using macros that operated on the raw movie associated with a single stimulus. These movies were acquired with 8-bit resolution. The first video frame in the sequence was thresholded to define the footprint but reject the surround. The value of this threshold was checked visually by the experimenter by highlighting the pixels above the threshold. Next, all the pixels in the largest contiguous region defined by this threshold were counted. The same threshold value was used for the area measurement in all frames of a stimulus movie. Examples of footprints defined and measured in this way are shown in FIG. 1D. The frames within the raw movie were not altered during this procedure.

A convenient way of visualizing the change in the area of the footprint was to make a “difference movie” by averaging 25 video frames from the beginning of a sequence and subtracting this “resting” image from all the individual frames in the sequence. Difference movies were made by converting the raw 8-bit movies to 64-bit floating point, thereby allowing pixels to have negative as well as positive values. In a difference movie, areas of no change appear grey with pixel values of zero, while areas where the footprint expands appear darker with negative pixel values (FIG. 1D). Areas where the membrane elevates from the surface appear brighter and have positive values (FIG. 6C).

The average area of the footprint of a goldfish bipolar cell terminal was 28.7 μm². The total surface area of the average terminal is about 388 μm², calculated from an average terminal capacitance of 3.1 pF (Neves and Lagnado, 1999), assuming a specific membrane capacitance of 8 fF μm⁻². Thus the footprint sampled only about 7% of the total surface area. The terminals of mouse bipolar cells were flatter against the coverslip and we estimated that the average footprint sampled about 30% of the surface area (see text).

Combination of IRM with TIRFM

An Axiovert 100 inverted microscope (Zeiss) was modified for “through-the-objective” TIRFM by removal of the normal epifluorescence condenser (Axelrod, 1989; Axelrod, 2001). Illumination was provided by the 488 nm line of an Argon Ion laser (100 mW, Melles Griot) coupled through a single-mode fibre (Point-Source). The beam was expanded 10×(Edmund Optics) and then reflected through the epifluorescence port of the microscope by a mirror at angle of 45° that reflected the beam in parallel with the optical axis of the microscope. The beam was focused to the back focal plane of a 1.45 NA 60×oil-immersion objective (Olympus Optical Co.) by means of a convex lens (f=400 mm). Translation of the mirror altered the radial position of the laser beam in the back focal plane of the objective and, therefore, the angle at which it left the objective. Positioning the beam at the periphery of the objective caused it to suffer total internal reflection at the interface between the coverslip and aqueous medium. The length-constant of the evanescent field was calculated as ^{˜}100 nm assuming a cell refractive index of 1.38.

To use TIRFM and IRM simultaneously, a combining cube (Edmund Optics) was placed in the optical path, after the focusing lens. This cube combined the laser beam for TIRFM with the collimated beam from a xenon lamp for IRM. The IRM beam was transmitted through a band-pass filter (535DF35; Omega Optical). The dichroic mirror in the filter block of the microscope (490-600 DBDR; XF2042, Omega Optical) reflected ^{˜}90% of the TIRFM beam at 488 nm and ^{˜}50% of the IRM beam at 535 nm. This mirror also transmitted >80% at wavelengths longer than 630 nm, allowing the collection of the red fluorescence of FM4-64. The green IRM image and red TIRFM image were magnified 3× and projected onto the CCD of a Princeton Instruments Pentamax Intensified camera (Roper Scientific). To allow simultaneous collection of the two images, an image splitter (Cairn Optosplit) separated the red and green images using a dichroic mirror (595DCXR, Chroma Technology Corp.) and filters (HQ535/50 for green channel and HQ665LP for red). A weak convex lens (f=630 mm) was placed in the red channel to make it parfocal with the green channel.

Images were acquired at 30 Hz using Winview 32 v2.5.A acquisition software (Roper Scientific). Image analysis was later performed using IPLab (Scanalytics) and Igor Pro (WaveMetrics). The shift required to align TIRFM and IRM images in the red and green channels was measured using 100 nm diameter fluorescent beads double-labelled red and green (Tetraspeck, Molecular Probes). Image acquisition was synchronized with electrophysiological recording by triggering using a Master-8 pulse generator (AMPI) which also controlled the shutter in the laser path. A camera exposure signal time was digitized along with the membrane current using PClamp 8.0 software (Axon Instruments, CA, USA) to record the timing of each frame relative to electrophysiological signals.

For imaging granules with TIRFM, chromaffin cells were incubated with the membrane dye FM4-64 (10 μM, Molecular Probes) for 18 to 48 hours and washed for at least 30 min prior to the start of the experiment. Although acridine orange is often used to label granules in chromaffin cells (Steyer et al., 1997), we preferred to label with FM4-64 for two reasons. First, we needed a dye that emitted at long wavelengths that could be separated from green light used for IRM. Second, granules labelled with acridine orange had a tendency to fuse spontaneously when exposed to the laser used for TIRFM illumination and this problem was not encountered when granules were labelled with FM4-64. Incubation with FM4-64 labelled fewer granules than acridine orange, perhaps because it partitioned into the most recently synthesized granules.

Cell Preparation and Electrophysiology

Depolarizing bipolar cells were isolated from the retina of goldfish by enzymatic digestion, as described previously (Burrone and lagnado, 1997; Neves and Lagnado, 1999), and plated onto borosilicate glass coverslips coated with poly-L-lysine. Cells formed footprints against the glass within about 20 mins, after which the chamber could be continuously perfused. Depolarizing bipolar cells from the retinae of C57 mice were isolated from animals at P28-P36 following the protocol described by Kaneko et al. (1989) and plated in the same way. Bovine chromaffin cells were prepared by the methods of Seward and Nowycky (1996).

Whole-cell patch clamp and capacitance recordings were performed at room temperature from the soma of retinal bipolar neurons using methods described by Neves and Lagnado (1999). The Ringer's solution for neurons from goldfish contained the following (in mM): 120 NaCl; 2.5 KCl; 1 MgCl₂; 2.5 CaCl₂; 10 glucose; 10 HEPES; pH 7.3; 280 mOsm/Kg⁻¹. The Ringer's solution for neurons from mouse contained: 105 NaCl; 10 TEACl; 2.5 CsCl; 1 MgCl₂; 5 CaCl₂; 1.25 NaH2PO₄, 26 NaHCO₃; 10 glucose; pH 7.42 with 95% O₂/5% CO₂; 300 mOsm/Kg⁻¹. The Ringer's solution for bovine chromaffin cells contained: 140 NaCl; 2.5 KCl; 1 MgCl₂; 2.5 CaCl₂; 10 glucose; 10 HEPES; pH 7.42; 300 mOsm/Kg⁻¹. Pipette solution for recording from goldfish bipolar cells contained 110 Cs-methane sulphonate; 10 TEACl; 5 MgCl₂; 3 Na₂ATP; 1 Na₂GTP; 20 HEPES; 0.4 BAPTA; pH 7.2; 260 mOsm/Kg⁻¹. In some experiments the BAPTA concentration was increased to 10 mM (as indicated in text). For current-clamp experiments, the electrode solution contained 120 mM KCl instead of Cs-methane sulphonate and TEA-Cl. Intracellular solutions for both mouse and bovine chromaffin cells contained: 120 Cs-methane sulphonate; 10 TEACl; 5 MgCl₂; 3 Na₂ATP; 1 Na₂GTP; 20 HEPES; 0.4 BAPTA; pH 7.2; 275 mOsm/Kg⁻¹. Recombinant light chain of botulinum toxin E was prepared as described by Vaidyanathan et al. (1999) and dialyzed against the intracellular solution before dilution to a concentration of 500 nM.

Capacitance measurements made with a patch-pipette on the cell body can underestimate capacitance changes in the terminal if the resistance of the axon connecting the two compartments is too high. To minimize this problem we used cells with short fat axons (estimated resistance less than about 20 MΩ) and sinusoidal command signals at frequencies lower than a few hundred Hertz (Mennerick and Matthews, 1997). In the sample of 8 cells we used for combined IRM and capacitance measurements, the average capacitance response to a 500 ms depolarization was 160 fF (FIG. 2B), which was about 50% higher than that measured in detached terminals by Mennerick and Matthews (1996) and Neves and Lagnado (1999). The axonal resistance does not alter the relative time-course of the fall in capacitance after a stimulus, and does not, therefore, affect measurements of the rate of endocytosis shown in FIG. 2B.

Results

Exocytosis in a Giant Synaptic Terminal Monitored by IRM

IRM was introduced into cell biology by Curtis (1964). The principles of IRM and the implementation we have used are shown in FIGS. 1A and B and described in the Experimental Procedures section above. FIG. 1C shows a transmitted light image of the giant terminal of a bipolar cell isolated from the retina of a goldfish and an IRM image of the same terminal. The dark “footprint” is the area where the close proximity of membrane to glass caused reflected light to undergo destructive interference. Changes in the area of the footprint caused by depolarization and calcium influx are shown in FIG. 1D as a series of “difference images” obtained by subtracting an averaged resting image from each subsequent video frame. The stimulus was a 0.5 s depolarization to −10 mV, a potential at which the calcium current was activated maximally. The difference image immediately before stimulation was uniformly grey (FIG. 1Da), but depolarization caused the appearance of a dark area due to the expansion of the footprint and the approach of membrane to the coverslip (FIG. 1Db). After the closure of calcium channels, the footprint retracted (FIG. 1Dc). The changes in area were quantified from the raw images using a simple algorithm that counted the number of pixels in the footprint that fell below a threshold (see the Experimental Procedures section above). FIG. 1Dd shows the outlines of the footprint calculated in this way for frames a, b and c. The full time-course of the changes in the area of the footprint is shown in FIG. 1E.

The averaged change in the area of the footprint triggered by a 500 ms depolarization is shown by the thick trace in FIG. 2A (n=19). The patch pipette contained 0.4 mM BAPTA as the Ca²⁺ buffer, because this roughly mimics the effects of the endogenous buffers (Burrone et al., 2002). The thin broken trace in FIG. 2A shows that increasing the concentration of BAPTA to 10 mM completely blocked the increase in area triggered by depolarization, demonstrating that the changes were due to an internal action of Ca²⁺. To investigate whether this action of Ca²⁺ was the stimulation of exocytosis, we carried out similar experiments with patch pipettes containing 500 nM of the light chain of botulinum toxin E (BoNT-E), a metalloprotease that inhibits exocytosis by selectively cleaving SNAP-25 and preventing formation of a normal SNARE complex (Xu et al., 1998; Schiavo et al., 2000). The thin trace in FIG. 2A shows that BoNT-E in the presence of 0.4 mM BAPTA reduced the expansion of the terminal by about 95%. These results demonstrate that expansion of the footprint in response to depolarization was due to exocytosis triggered by Ca²⁺.

Comparison of IRM Measurements of Exocytosis and Endocytosis with Capacitance and FMI-43 Measurements

Changes in the area of the footprint were compared with changes in total surface area of the terminal by simultaneous use of the IRM and capacitance techniques, using a patch pipette on the cell body (see the Experimental Procedures section above). The thick trace in FIG. 2B is the average change in the area of the footprint elicited by a 500 ms depolarization and the thin trace is the averaged capacitance response measured in the same group of 8 cells. Retraction of the footprint occurred with a time-course very similar to endocytosis measured by capacitance.

Applying the same 500 ms depolarization to terminals detached from the rest of the cell, Neves and Lagnado (1999) measured an average capacitance response of 105 fF in a group of terminals with a resting capacitance of 3.1 pF, yielding a relative increase in surface area of 3.4%. The results in FIG. 2B are in close agreement; the increase in the area of the footprint averaged 3.3%. The absolute amplitude of the capacitance response in this group of cells was 160 fF, about 50% larger than that measured in isolated terminals. The size of the terminals used in the present study may have been larger because we purposefully selected cells with large footprints so as to measure area changes with better signal-to-noise.

Changes in surface area measured by IRM and capacitance did not always show good agreement within individual cells, but this was not unexpected because the average footprint represented only 7% of the surface area of a terminal (see the Experimental Procedures section above), and therefore sampled exocytosis from an average of only 2-3 active zones from a total of about 34 (Llobet et al., 2003).

Changes in the area of the footprint measured by IRM were dependent on the duration of the stimulus. FIG. 3A shows a single response to a 20 ms depolarization to −10 mV, a stimulus that completely depletes the rapidly-releasable pool of vesicles (Mennerick and Matthews, 1996; Gomis et al., 1999). The area of the footprint increased by 4% within two video frames (80 ms) and then immediately recovered to baseline with a time-constant of 1.1 s. The amplitude and kinetics of this response were similar to relative changes in the area of whole terminals measured using the capacitance technique (Mennerick and Matthews, 1996; Neves and Lagnado, 1999). In particular, the time-course over which the area of the footprint declined after this brief stimulus was similar to the rate of fast endocytosis measured by the capacitance technique (von Gersdorff and Matthews, 1994; Neves and Lagnado, 1999; Neves et al., 2001).

Increasing the duration of the depolarization to 5 s demonstrated that the expansion of the footprint occurred in three distinct phases. FIG. 3B shows an individual example in which the area of the footprint increased 1.3% in the first 40 ms, followed by a steady increase of 1.5% s⁻¹ over 2.5 s, then a continuous slow increase at a rate of 0.22% s⁻¹. Three phases of exocytosis have also been observed by rapidly staining vesicular membrane with FM1-43 (Neves and Lagnado, 1999). Another distinctive feature of the response to a longer stimulus was the continued slow increase in the area of the footprint for a few seconds after the closure of Ca²⁺ channels (FIGS. 2 and 3B). Again, a period of asynchronous exocytosis driven by residual Ca²⁺ is also a feature of responses measured using the capacitance technique or FM1-43 (Neves and Lagnado, 1999; Zenisek et al., 2000; Neves et al., 2001).

The averaged IRM response to a 5 s depolarization is shown by the thick trace in FIG. 3C, where it is compared with FM1-43 measurements of exocytosis measured in another group of cells (thin trace). The FM1-43 signals were normalized to the fluorescence of the plasma membrane, measured with the dye in the medium before stimulation (Neves and Lagnado, 1999). The FM1-43 and IRM measurements agreed closely at short times after the beginning of depolarization, but at later times the signals diverged. First, the FM1-43 signal continued to rise while the IRM signal tended to plateau. Second, the FM1-43 signal remained steady after closure of Ca²⁺ channels while the IRM signal declined. These observations are consistent with the results of Neves and Lagnado (1999), who compared FM1-43 and capacitance signals and also found that they diverged within 1-2 s of beginning stimulation. The FM1-43 signal is the time integral of exocytosis because vesicular membrane remains stained whether it is at the surface or has been retrieved by endocytosis. But IRM, like capacitance, measures the net difference between exocytosis and endocytosis. The divergence between FM1-43 and IRM signals is therefore due to membrane retrieval occurring during stimulation. FM1-43 and capacitance signals have been compared to provide real-time measures of endocytosis in chromaffin cells (Smith and Betz, 1996) and bipolar cells (Neves and Lagnado, 1999).

The results in FIG. 1-3 demonstrate that exocytosis and endocytosis in the synaptic terminal of goldfish bipolar cells cause changes in the area of the footprint measured by IRM, and these changes agree quantitatively with measurements of exocytosis and endocytosis made using the capacitance technique and the membrane dye FM1-43.

Exocytosis Measured During Electrical Activity

Capacitance measurements cannot be made from whole-cell recordings while voltage-sensitive conductances, such as Ca²⁺ channels, are active (Gillis, 1995). FIGS. 1-3 demonstrate that IRM can overcome this drawback, providing a continuous readout of changes in surface area during stimulation. This advantage of the IRM technique allowed us to investigate exocytosis in bipolar cells while their intrinsic conductances were active. This is of interest because few studies of exocytosis by these neurons have investigated the properties of exocytosis at physiological membrane potentials. In the retina, the effect of light is to inject current into depolarizing bipolar cells by causing the opening of cation channels in the dendrites. L-type Ca²⁺ channels activate around −43 mV but the strongest lights do not depolarize these neurons beyond −30 to −25 mV because of a Ca²⁺-activated K⁺ conductance (Ashmore and Falk, 1980; Burrone and Lagnado, 1997). We mimicked the effect of light by making current-clamp recordings with K⁺ conductances active. FIG. 4A shows changes in the area of the footprint in response to current steps of +2 pA and +20 pA, both lasting 5 s. The membrane potential during the 20 pA step averaged −28 mV, and this caused twice the peak increase in area triggered by the 2 pA step, which depolarized the cell to −29 mV. Thus a depolarization of just 1 mV over this voltage range caused a large increase in the amount of exocytosis, demonstrating the exquisite sensitivity of this ribbon synapse (Burrone and Lagnado, 2000).

A comparison of exocytic response at physiological potentials with that at −10 mV is shown in FIG. 4B. The averaged response to a +2 pA step (thin trace) caused an increase in surface area of about 1.5%, indicating that endocytosis quickly balanced exocytosis. The rapid phase of release at −10 mV (thick trace) was about three times the size of the initial response to the current step and the second phase of release was 10-times faster. Endocytosis did not balance exocytosis until the net increase in surface area had reached about 9%. These results demonstrate that normal rates of exocytosis at the terminal of bipolar cells are far below the maximum that this ribbon synapse can support.

Exocytosis and Endocytosis in the Synaptic Terminal of Mouse Bipolar Cells

Neurons isolated from mammals tend to have small synaptic terminals and complex morphologies, making it difficult to study exocytosis and endocytosis using the capacitance technique. One recent exception is the study of Pan et al. (2001), in which the capacitance technique was used to detect exocytosis in isolated terminals of rat bipolar cells. To test the possibility of using IRM to make real-time measurements at a mammalian synapse, we used bipolar cells isolated from the retina of mouse. The terminals were typically 3-4 μm across (FIG. 5A), but yielded clear footprints, often sending out a number of short processes (FIG. 5B). FIG. 5B shows examples of “difference” images obtained before, during and after a 500 ms depolarisation from −70 mV to −20 mV. The time-course of the change in area is shown in FIG. 5C. A notable feature of this response was the large increase in surface area that occurred after the end of the stimulus, indicating asynchronous release driven by residual Ca²⁺. Membrane retrieval was complete over a period of 10 s.

The exocytic response to a 500 ms depolarization averaged from 17 terminals is shown in FIG. 5D. The peak increase in area was about 2.9%, which is comparable to the relative increase in capacitance measured by Pan et al. (2001) in rat bipolar cells. Using IRM we were also able to monitor the increase in area occurring during depolarization. There appeared to be two phases of exocytosis: an area increase of ˜0.5% occurred within 40 ms, followed by a steady increase at a rate of 2.24% s⁻¹ for about 1 s (thick straight line in FIG. 5D). Thus mouse bipolar cells contain at last two vesicle populations that are released at different rates in response to depolarization. We estimated the size of the rapidly-releasable pool (RRP) as 0.5% of the resting surface area by extrapolating the linear phase of area increase back to the start of the stimulus. The average area of these footprints was 12.1±1.6 μm², so the 0.5% increase caused by release of the RRP represented an area of about 0.06 μm², which we is equivalent to ˜9 vesicles with a diameter of 46 nm. A spherical terminal with a diameter of 3.8 μm would have a surface area of 35 μm², so the average footprint was roughly 30% of the total membrane area. We therefore estimate that the rapidly-releasable pool (RRP) in mouse bipolar cells is composed of about 30 vesicles, while the slow phase of exocytosis occurred at a rate of ^{˜}190 vesicles/s. Both these estimates would be doubled if vesicles were only 30 nm in diameter. A useful application of IRM may be as a real-time assay for endocytosis at small synaptic terminals inaccessible to the capacitance technique.

Fusion of Individual Granules Visualized in Chromaffin Cells

Neuroendocrine cells and related cell lines are widely used in the study of Ca²⁺-triggered secretion (Bader et al., 2002; Barg et al., 2002). Although the organelle fusing with the surface membrane is a dense-core granule about 300 nm in diameter, these cell types have provided a number of important insights into the function of proteins that are also involved in exocytosis at the synapse (Rettig and Neher, 2002). We therefore investigated the use of IRM to study exocytosis in bovine chromaffin cells. FIG. 6A shows the footprint of a chromaffin cell together with “difference” images obtained before, during and after a 500 ms depolarization from −80 mV to +10 mV. The most striking effect of stimulation was the appearance of discrete bright spots within the footprint, indicating local regions of membrane that were raised from the surface of the coverslip. The time-course of the intensity change in the center of two of these spots is shown in FIG. 6B. The signal reached a maximum 80-120 ms after its onset and then declined to baseline over the next few seconds. The mean delay between the start of depolarization and the intensity peak in these spots was of 310±4 ms (range from 70 ms to 1.82 s, n=61). On average, a 500 ms stimulus triggered the appearance of 6.6±0.7 spots in footprints with an area of 49 ±8 μm² (n=11), or about 0.13 events per μm².

We tested how the bright spots that appeared under IRM might be related to secretion by simultaneously imaging individual fusion events using TIRFM. Granules were labelled with FM4-64 (see the experimental Procedures section above) and fusion events were detected as the “burst” of fluorescence caused by diffusion of the dye into the footprint (Steyer et al., 1997; Zenisek, 2002). A depolarization from −80 mV to +10 mV lasting 500 ms caused discrete bursts of fluorescence in the TIRFM image that superimposed in space and time with the appearance of bright spots under IRM. An example of spatial colocalization from a footprint in which the stimulus triggered fusion of just one granule is shown in FIG. 6C. The temporal correlation between fusion of the granule and appearance of the bright spot in the IRM signal is shown in FIG. 6D. Of 15 fusion events detected by the release of FM4-64, 14 were associated with the appearance of a bright spot in the IRM image (6 cells). We conclude that the IRM technique provides a direct method for detecting the place and time of granule fusion in neuroendocrine cells.

The capacitance response triggered by a 500 ms depolarization was correlated with the density of granules fusing in the footprint of the same cell, as shown in FIG. 7A. In FIG. 7B the same results are used to plot the capacitance change against an estimate of the total number of fusion events in the cell, obtained by multiplying the number of bright-spots per unit area by the total surface area measured from the resting capacitance (assuming a specific membrane capacitance of 8 fF μm⁻²). The slope of the line through the points is 1.43 fF/granule, which is in close agreement with the average capacitance of chromaffin cell granules measured in cell-attached patches (Ales et al., 1999). These results provide further support for the conclusion that bright spots appearing in IRM images were caused by the fusion of individual granules. Thus the quantification of triggered exocytosis from secretory granules can be carried out by counting bright spots appearing within the footprint. Unlike synaptic terminals, changes in the area of the footprint did not correlate with changes in total membrane area measured by capacitance (FIG. 7D). Thus the most direct measure for granule fusion was the increase in intensity caused by the loss of destructive interference. But it should be noted that the IRM signal from chromaffin cells also showed a slower change, occurring with a delay after granule fusion; this change was a decrease in the area of the footprint associated with strong responses reflecting detachment of the membrane at the edges of the footprint from the substrate (FIG. 7C).

Discussion

We have described how IRM can be used to provide a real-time assay of exocytosis and endocytosis at synapses and neuroendocrine cells with examples from three different preparations: a neuron from the goldfish retina with a giant synaptic terminal, a neuron from the mouse retina with a small synaptic terminal and bovine chromaffin cells. IRM provides detailed information about the kinetics of exocytosis and endocytosis, comparable to other real-time methods such as the capacitance technique or fluorescent membrane dyes (Angleson and Betz, 1997). Advantages of IRM over the capacitance technique are that it provides a continuous readout of exocytosis, does not require the cell to be voltage-clamped and can be applied to small neurons with complicated morphologies. Like TIRFM, IRM allows the visualization of fusion by individual granules in neuroendocrine cells, but at a fraction of the cost and without fluorescence photodamage.

We have also demonstrated how IRM can be used to assay the inhibition of exocytosis and endocytosis, with examples using two agents: calcium buffers (that suppress the calcium signal triggering exocytosis) and botulinum toxin (that interferes with the protein machinery involved in exocytosis of vesicles and granules).

Exocytosis of granules can also be triggered in cell-free systems of cell membranes attached to a substrate. As an example, these membranes can be obtained from PC12 cells, a cell line that secretes catecholamines from dense-core granules (Avery et al., 2000; Holroyd et al., 2002). The membranes stay attached to the substrate after the remainder of the cell on the substrate is destroyed by, for instance, sonication. Granules stay attached to the membrane after the cell is disrupted and can fuse on stimulation (Holroyd et al., 2002). The results shown above indicate that our use of IRM to detect fusion of secretory granules will also operate in such cell-free systems.

Rapid Collapse of Small Vesicles and Slow Collapse of Large Granules

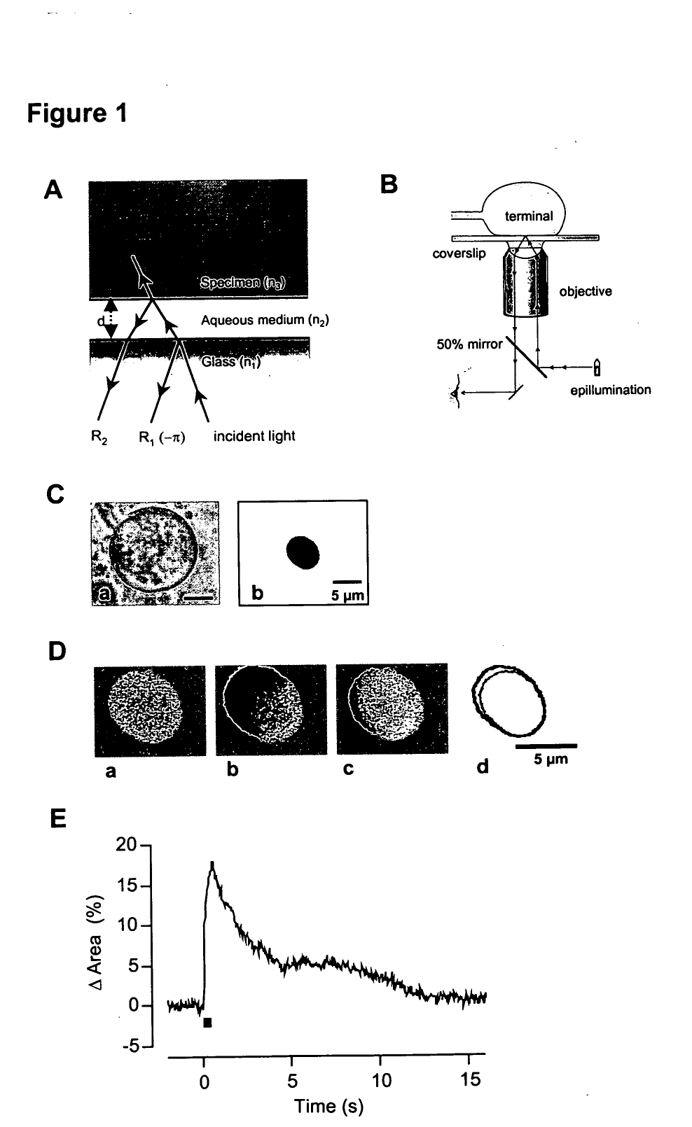

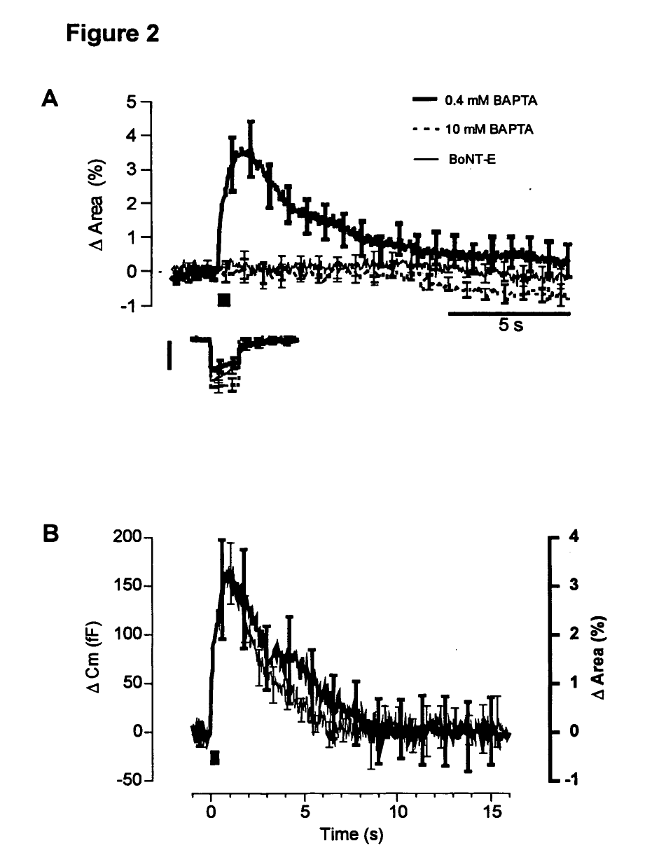

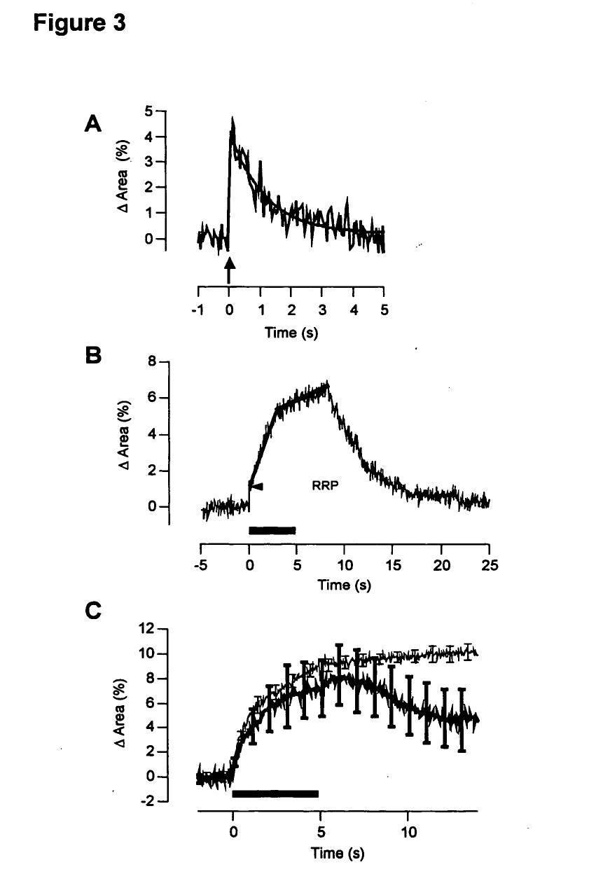

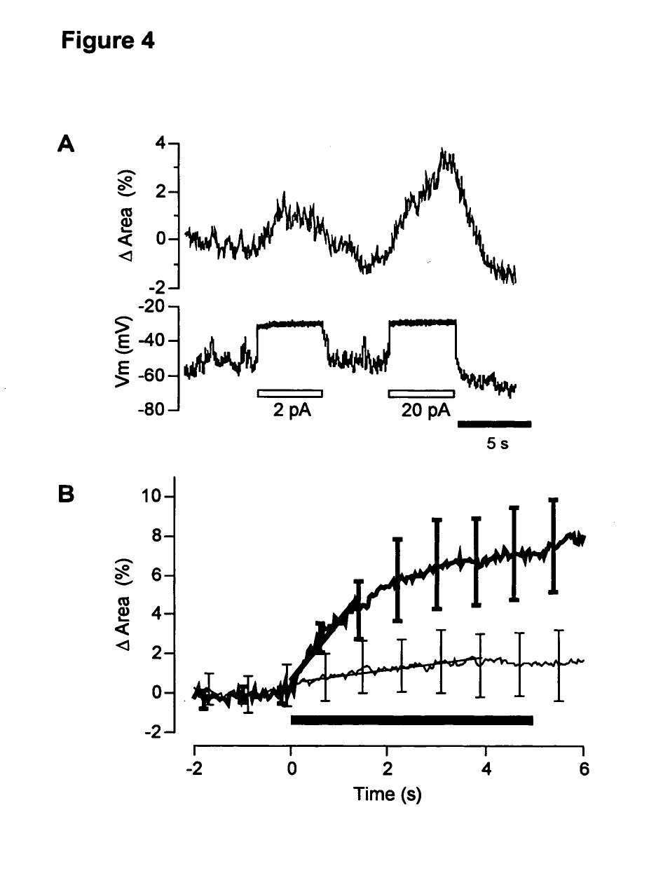

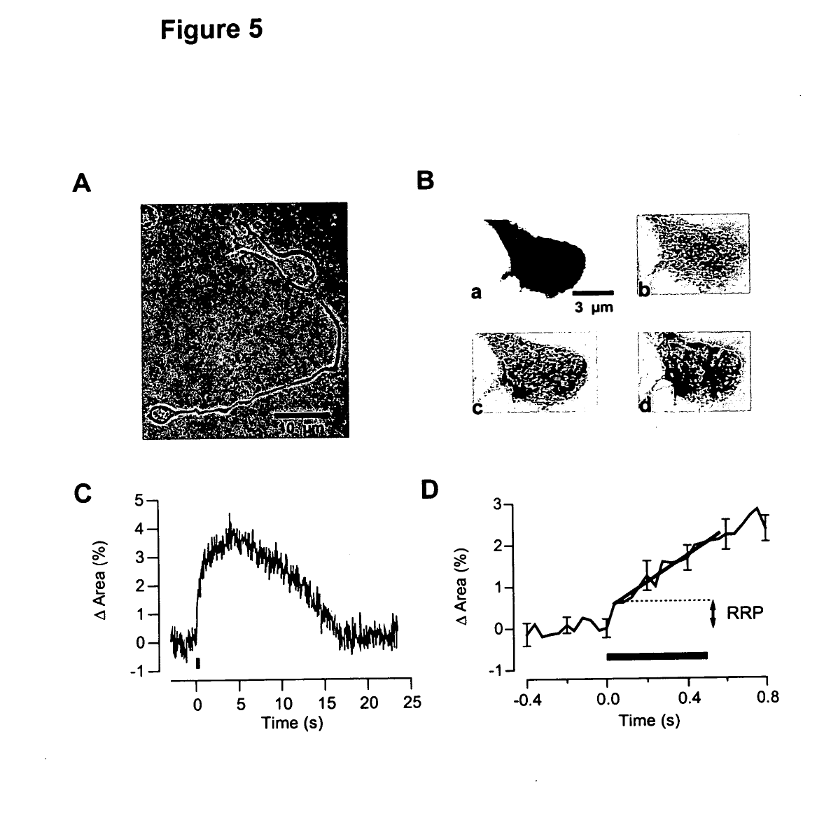

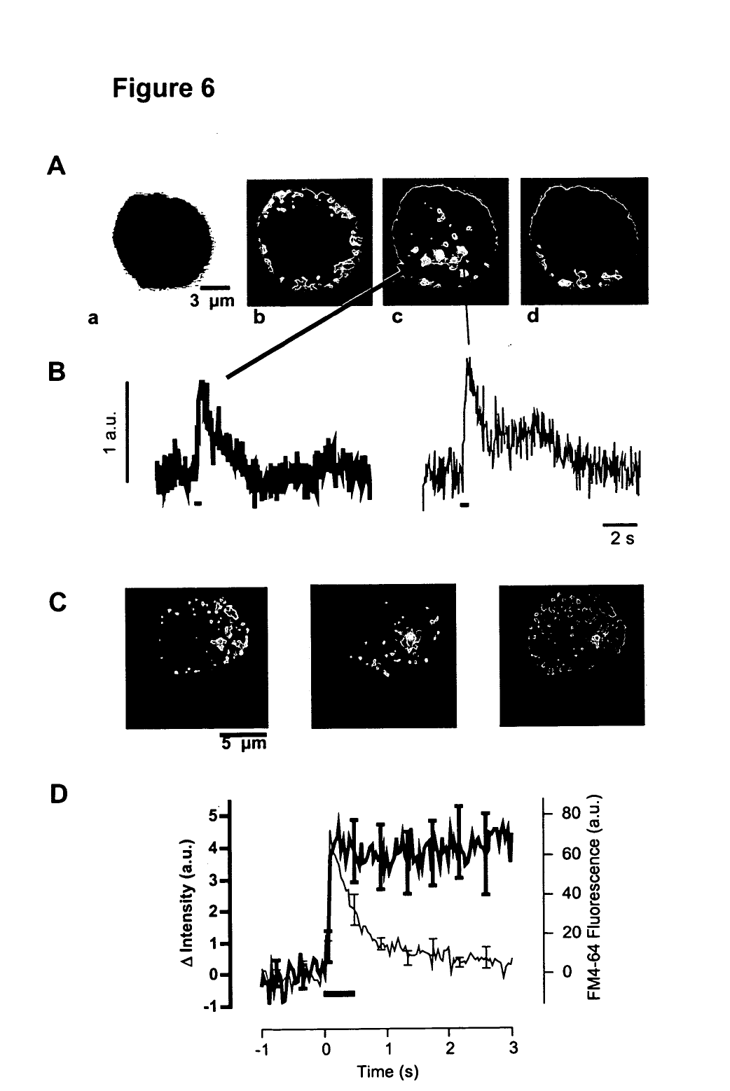

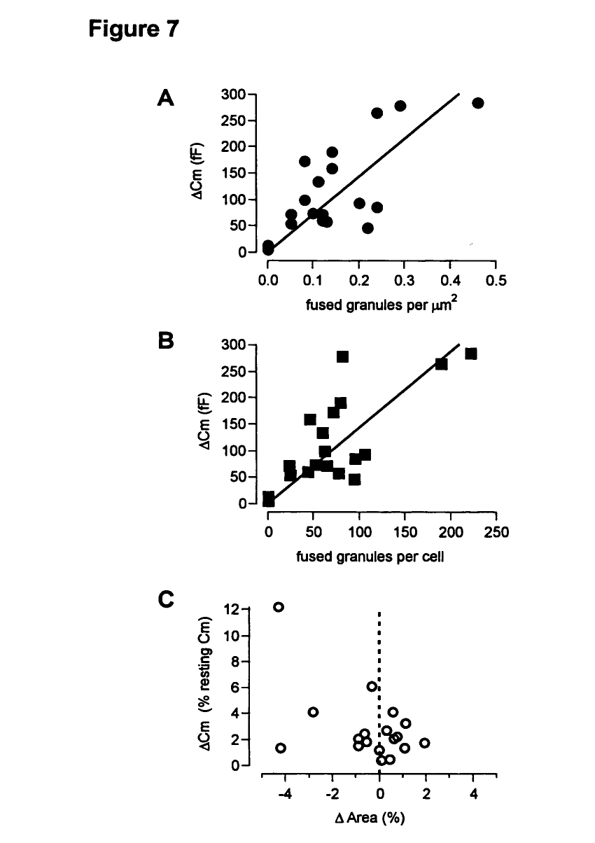

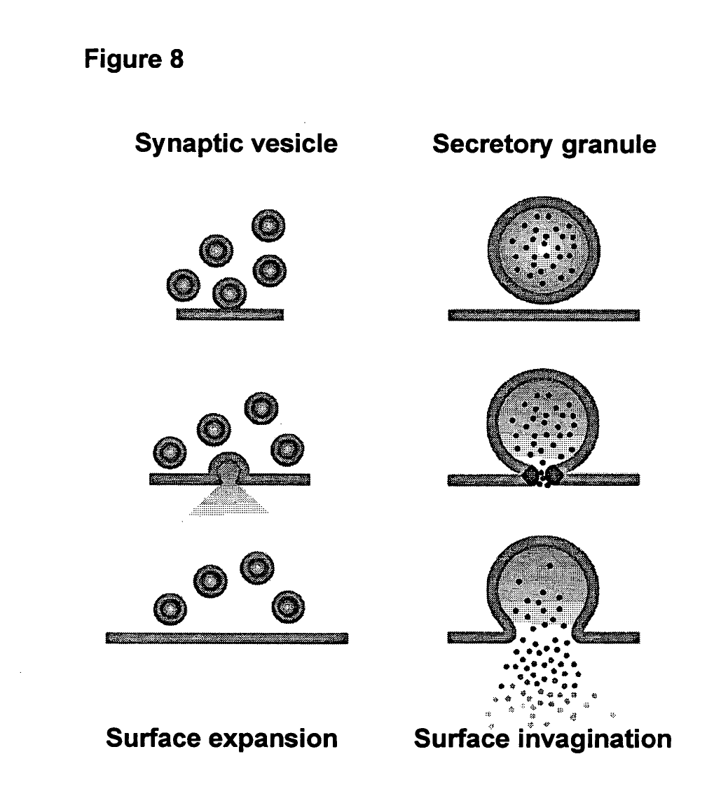

IRM can measure changes in the distance between the surface membrane and substratum of the order of tens of nanometers (Izzard and Lochner, 1976; Gingell and Todd, 1979; Weber, 2003). This resolution revealed a fundamental difference in the way in which small synaptic vesicles and large granules fuse with the surface membrane. Exocytosis in synaptic terminals caused membrane to spread across glass within tens of milliseconds, indicating that vesicles rapidly collapsed into the surface (FIGS. 1-5). In contrast, exocytosis in chromaffin cells caused the appearance of discrete surface invaginations that remained elevated for seconds at the site of fusion, indicating that the collapse of granules was much slower (FIG. 6). Evidence that granules form a long-lived membrane invagination after fusion has also been obtained by imaging a cytoplasmic marker using TIRFM: at sites of granule fusion the marker is excluded from spots in the evanescent field close to the coverslip (Taraska et al., 2003). This difference in the behaviour of small vesicles and large granules is shown schematically in FIG. 8. The slow decline in the IRM signal at sites of granule fusion in chromaffin cells (FIG. 6B) reflects the time-course of endocytosis measured by capacitance.

There is evidence that synaptic vesicles do not always collapse into the surface membrane when exocytosis is stimulated (Klyachko and Jackson, 2002; Gandhi and Stevens, 2003; Aravanis et al., 2003). Mechanisms by which vesicles communicate with the external medium while remaining intact are generally termed kiss-and-run”, and these are thought to involve exocytosis through a fusion pore that opens transiently (Valtorta et al., 2001). Vesicles communicating with the outside through a fusion pore are still expected to generate a capacitance increase (Albillos et al., 1997), but comparison of capacitance and IRM measurements in bipolar cell terminals indicated that most or all exocytosis in the footprint resulted in membrane expansion as well as a capacitance increase (FIG. 2B). These results therefore indicate that most vesicles in the synaptic terminal of bipolar cells rapidly collapse with the surface membrane. This conclusion is consistent with recent work using TIRFM demonstrating that FM1-43 in the vesicle membrane rapidly mixes with the lipids of the surface following fusion (Zenisek et al., 2002). The relative importance of exocytosis by “kiss-and-run” as opposed to full collapse may depend on the synapse in question. For instance, Gandhi and Stevens (2003) found that “kiss-and-run” was much less prevalent at hippopcampal boutons with high release probability compared to those with low.

Further Investigation of IRM as a Method to Study Secretion

Basic aspects of the IRM signals associated with exocytosis and endocytosis deserve further investigation. For instance, the footprints of bipolar cell terminals did not expand uniformly when exocytosis occurred. Rather, expansion tended to localize to one or two areas. In the case of mouse bipolar cells, responses were commonly observed in the processes formed by the terminal. An intriguing possibility is that these localized responses reflect the distribution of active zones within the footprint. This idea might be investigated by combining TIRFM and IRM to localize sites of Ca²⁺ influx, vesicle fusion and ribbons (Zenisek et al., 2003). It will also be interesting to investigate whether IRM might give insights into the process of vesicle fusion or retrieval. The present results indicate that small vesicles undergoing exocytosis collapse into the surface membrane within 40-80 ms, but imaging at higher time-resolution with cameras of higher dynamic range might allow the detection of “omega”-shaped deflections of the surface membrane associated with exocytosis and/or endocytosis (Koenig et al., 1998). Of course, “omega” shapes the size of small synaptic vesicles will generate smaller signals than granules in chromaffin cells.

High-Throughput Assays

A central aspect of the function of the immune, endocrine and nervous systems is the transmission of signals by the secretion of chemical agents that act on other cells. Inappropriate release of these agents is responsible for an enormous range of diseases. One important example is diabetes mellitus, a major cause of which is underproduction of insulin by pancreatic beta cells. Among the most important drugs used to treat diabetes are the sulphonylureas that act on beta cells to stimulate secretion of insulin. Other important examples are the hypersensitivity reactions caused by the release of histamine and leukotrienes from granules in mast cells. These reactions include allergic asthma, hay fever, drug allergies, uriticaria and systemic anaphylaxis. Allergies and asthma are often treated using a class of drug called mast cell stabilizers (e.g cromolyn salts and neodocromil) that prevent mast cell degranulation.

The search for effective drug-based treatments of diseases involving the release of chemical mediators is hampered by the lack of assays for secretion that are amenable to high-throughput screening. The usual biochemical assays of secretion from cells of the immune, endocrine and nervous systems are slow and cumbersome. An easy and efficient assay for secretion would also greatly aid research into the steps involved in secretion and, therefore, the identification of potential drug targets.

Thus, advantageously, the method of the present invention can be used to make real-time measurements of secretion from cells of the nervous, endocrine or immune systems (including related cell lines). We have demonstrated that the method works for detecting secretion of neurotransmitters and hormones. The method can be performed simply, directly and relatively cheaply.

Changes in the intensity of reflected light are caused by deflections of the surface membrane associated with the secretion process itself. We have demonstrated that secretion of neurotransmitters contained in synaptic vesicles causes more destructive intereference, whereas secretion of hormones contained in large dense-core granules causes less destructive interference (FIG. 8).

The method does not depend on detection of a chemical that is released from the granule, or contained in the granule or present in the granule membrane. Rather, secretion can be detected from any one of the large number of cell types in which signalling molecules are contained in vesicles, granules or other similar organelles.

There are already machines on the market that can perform high-throughput screening of cell-based assays. These assays are almost all based on detecting a fluorescent signal, usually reflecting the biological activity of a single molecular species in the cell. These machines can be split into two categories, those that operate on cell populations (collecting the signal from a large number of cells in a single well on the plate) and those that image individual cells or small groups of cells in the well.

The first category do not require high-quality imaging apparatus or sophisticated cameras: the fluorescence might be collected by a photomultiplier tube to give the signal averaged over many cells. Examples of such machines are the FLIPR from Molecular Devices, and VIPR from Aurora Instruments.

The second type of machine uses imaging apparatuses similar to high-quality research microscopes, as well as high-quality cameras. However, a problem associated with the use of fluorescent probes is that the cameras must be sensitive (and therefore e.g. incorporate cooled CCDs), and tend to be expensive. A further problem is that collection of enough light to generate decent images takes significant time. Examples of available machines designed for cell-based high-throughput screening where individual cells are imaged are the IN Cell Analyzer 3000 from Amersham Biosciences, the Discovery 1 from Universal Imaging Corporation and the ImageXpress from Axon Instruments. These machines image just one well at a time (i.e. they are equivalent to a single microscope with a high-quality camera attached).

According to the present invention, IRM can also be adapted to perform high-throughput screens e.g. of secretion from cells in multi-well plates. Compared to fluorescence-based screens, IRM screens should be relatively simple to implement because the signal (light reflected back from a transparent substrate) is easy to produce and detect. Also an IRM screen can be used to interrogate individual cells from larger populations. For example, one possible strategy would be to measure secretion from cells in a multi-well plate in response to an appropriate stimulus (e.g. depolarization by application of high potassium concentration or application of specific agonist such as nicotine). Alterations of this response (inhibition or potentiation) would be measured when test compounds are also present. Compounds that enhance or inhibit secretion would thereby be identified.

A high-throughput, IRM-based, screening apparatus could be based around existing technologies, including e.g.:
High-density microplates containing cells of interest (e.g. a mast-cell line)
Robots that manipulate these plates on and off the imaging platform (e.g. Zymark Twister)
Automated liquid-handling systems that add a chemical that stimulates secretion (e.g. IgE that stimulates mast-cells) and/or test substances that might alter the secretory response (e.g. Zymark Sciclone ALH 3000)
Programmable computer systems for controlling the robots and automated liquid-handling systems, and for processing, displaying and storage of images and results

Preferably, however, such an apparatus also has means for automatically optically coupling the microscope objective lens to the microplate or other substrate to which the cells are attached. Such coupling is typically performed by a medium of appropriate refractive index (oil in the case of cells on a glass substrate). In a preferred embodiment, the multiwell plate will have a glass bottom to provide the required degree of flatness and optical clarity, as is commonly required in cell-based assays. Such plates are provided by, amongst others, Whatman; a 175 micron glass coverslip is incorporated into the bottom of each well of a 96-well plate. The cells are attached to the glass substrate, often within minutes of placing cells on the substrate. The coverslip may also be coated to enhance attachment of cells, for instance with poly-lysine or conconavalin A.

In a further embodiment, the cells may be plated on a plastic substrate (e.g polystyrene or acrylic). To perform IRM, the refractive index of the substrate on which the cell is attached is preferably closely matched to the refractive indices of the material from which the objective lens is constructed and the medium which couples the objective lens optically to the substrate. Plastic plates may be used in conjunction with objective lenses of plastic and immersion media of similar refractive index (e.g. solutions of sugar in water of the appropriate concentration).

The apparatus may also have means for automatically focusing the objective lens to the plane in which the cell membrane is attached to the substrate. Methods of auto-focusing using lasers are commonly employed in commercial machines used for cell-based assays, such as the ImageXpress from Axon Instruments.

In further embodiments, the apparatus may be coupled to a flash-lamp or similar light source that allows photolysis of “caged” compounds, such as the caged calcium NP-EGTA (Molecular Probes).

An IRM-based, high-throughput screen should be able to operate at high speed because it detects the secretory process as it happens, not the released chemical. It should also be possible to multiplex the collection of the signal from many wells simultaneously (which is equivalent to many microscopes operating in parallel) because the signals obtained by IRM tend to be brighter than those obtained from existing systems based on fluorescence detection, allowing the use of many small cheap cameras in parallel. Unlike fluorescence-based cell assays, the read-out is not a dim fluorescent signal, so expensive fluorescence detector cameras and complicated image processing are not required. The detector might be as simple as a black-and-white video camera. Indeed if a “global” IRM signal from a large population of cells on the substrate is merely wanted, a photodiode detector may be used in place of a camera.

Furthermore, no special reagents are required to detect secretion with IRM, whereas they are a major cost in fluorescence-based assays.

The method of the present invention assays the end-point of secretion processes (e.g. fusion of granules or vesicles), rather than the activity of the many proteins involved in regulating this process. This is significant because a screen based on the method allows a large number of possible drug targets to be tested for regulation of secretion. Most current cell-based assays use fluorescence to look at a single target involved in the cellular process of interest.

Furthermore, unlike biochemical assays that detect a particular secreted chemical, the method we have developed will be applicable to a very wide range of different cell types, secreting a wide range of different hormones, cytokines, inflammatory agents, neurotransmitters etc

Possible uses of an IRM-based, high-throughput screen are:

Research into the steps involved in secretion and, therefore, the identification of potential drug targets
Screening of libraries for compounds affecting secretion

For example, it might be of interest to screen for drugs that affect secretion of insulin from insulinoma cell lines and mast-cell degranulation. Other productive areas could be screening related to diseases involving the immune system, where secretion from granules in lymphocytes, mast cells, macrophages and eosinophils are key events.

Stimulation may be applied by a variety of methods, including; electrical stimulation using field electrodes in the medium containing the cells; direct patch-clamping of individual cells; application of substances such as potassium to depolarise cells; application of calcium ionophores such as A23187 to directly cause calcium influx into cells; hormones, antibodies, peptides, antigens, cytokines, growth factors, organic molecules, action potentials, or other cells (i.e. cell-cell contacts). Another method of stimulation is the use of “caged” calcium, such as NP-EGTA (Rettig and Neher, 2002), which can be loaded into cells and then release calcium after the cage is photolyzed by application of intense light. Usefully, NP-EGTA can be loaded into cells as the aceto-methoxy ester.

Candidate bioactive agents may be screened for the ability to modulate exocytosis or endocytosis. For example, the candidate bioactive agents may be combined with the cell population before, during or after exocytosis is stimulated. In some instances, it may be desirable to determine the effect of the candidate bioactive agent on a cell which is not specifically stimulated to undergo exocytosis or endocytosis or a cell in which exocytosis or endocytosis is inhibited. The candidate bioactive agent can be added to the cell population exogenously or can be introduced into the cells.

In preferred embodiments, the candidate bioactive agents are nucleic acids designed to effect silencing of gene products by RNA interference hereafter termed “RNAi” (Hannon (2002)). The nucleic acids may be single- or double-stranded polynucleotides of two or more bases and are designed against coding sequences for candidate genes as identified by database searches of available genomic sequences. The nucleic acids are double-stranded RNA molecules complementary to the messenger RNA for a gene; 21-nucleotide short interfering RNAs (siRNAs) (Elbashir (2001)); single stranded RNA molecules that form double-stranded “hairpins” termed short hairpin RNA molecules (shRNAs); libraries of dsRNA, siRNA or shRNA that are randomized to target a host genes or selectively designed to target all (or a subset) of genes in a given cell type, tissue or organism; or any other polynucleotide desgined to selectively suppress a gene target. The polynucleotides may be produced synthetically and directly transferred to the cells under study or generated inside the cell by expression of a plasmid DNA molecule possessing the appropriate sequences for expression of the above molecules.

In preferred embodiments, the invention provides methods for screening a population of cells for those with an altered phenotype. Such a cell population may be a cell line or a proliferated population, for instance of mast cells or basophil cells. The altered phenotype may be a decrease or an increase in the amount of exocytosis or endocytosis in one cell compared to another cell or in the same cell under different conditions. Cells with altered phenotypes may be analysed to determine the cause of altered exocytosis or endocytosis.

By assaying exocytosis and endocytosis using intereference reflection microscopy, it is possible to detect not only alterations in exocytosis or endocytosis, but also alterations of different steps of the exocytotic pathway. Multiple cellular steps involved in the regulation of exocytosis and endocytosis, and any one may be assayed by the present methods. For instance, exocytosis in many cell types is triggered by calcium entry through voltage-sensitive calcium channels and substances that block these calcium channels also block exocytosis (Rettig and Neher, 2002). Thus, assaying exocytosis also allows one to screen for substances that block calcium channels. A second example is shown in FIG. 2 and described above, where botulinum toxin E is demonstrated to block exocytosis of vesicles in bipolar cell terminals. This toxin is a metalloprotease that cleaves SNAP-25, a key molecule in triggered exocytosis (Schiavo et al., 2000). One skilled in the art will appreciate that exocytosis and endocytosis are cellular processes that are regulated by a wide-range of other events in the cell, and that modulation of these other events may result in altered exocytosis or endocytosis. Thus, screening methods based on monitoring exocytosis and endocytosis using IRM will allow the detection of bioactive agents acting on a wide range of cell molecules and processes.

Cell Secretion Mediated by Granules

This section lists and describes in more detail cell types in which granules mediate secretory events and which may be the subject of tests or high-throughput assays according to the present invention. In particular, bioactive agents which affect secretory function can be studied by such tests or assays. The wide variety of cells that secrete the contents of granules is reviewed by Burgoyne and Morgan (2003).

Endocrine Cells

(i) Posterior Pituitary Cells

These secrete granules (large dense-core vesicles) containing a number of hormones and neuropeptides (Senda, 1995). They play a central role in endocrine function. The posterior pituitary releases antidiuretic hormone (ADH) and oxytocin. Pituitary tumours (adenomas) can lead to diseases due to hypersecretion or hyposecretion of these hormones. Examples: ACTH, Cushing's disease; GH, Acromegaly, PRL, Amenhorrhea/Galactorrhea in women and Loss of libido/Impotence/Galactorrhea in men; TSH, Hyperthyroidism. Treatments: Prolactinomas—dopamine agonists (Bromocriptine); Acromegaly—somatostatin analogues (Octreotide); Cushing's disease—blockers of cortisol release (Metyrapone). Pituitary function is also involved in rheumatoid arthritis (Cutolo et al., 2003), stress (Carrasco and Van de Kar, 2003), and obesity (Cummings and Schwartz, 2003). A group of cell lines derived from the pituitary of different species also have similar properties: from rat (Rattus norvegicus): GH1, GH3, MMQ and from mouse (Mus musculus) AtT-20, AtT-20ins (CGT-6) and AtT-20/D16v-F2.

(ii) Hypothalamic Neurons

These secrete granules, as does the cell line GH4C1, generated from rat (Rattus norvegicus) hypothalamic tissue secretes prolactin; growth hormone (somatotrophin).

Neuroendocrine Cells

(i) Chromaffin Cells from the Adrenal Gland.

These are often termed “neuroendocrine cells” and are used by biologists as a so-called “model system” for the study of secretion from both granules and small synaptic vesicles (Rettig and Neher, 2002). They release adrenaline and noradrenaline and are involved in hypertension, which can be caused by tumours called pheochromocytomas (Parmer and Zinder, 2002). The PC-12 cell line generated from Rattus norvegicus(rat) adrenal tissue has similar properties of release to chromaffin cells.

(ii) Cells of the Anterior Pituitary

These release granules (large dense-core vesicles) containing a number of hormones and neuropeptides (Senda, 1995). They play a central role in endocrine function. The anterior pituitary releases: Adenocorticotrophic Hormone (ACTH), Growth Hormone (GH), Prolactin (PRL), Thyroid stimulating hormone (TSH), Folicular stimulating hormone (FSH), Lutenising hormone (LH), Melanocyte stimulating hormone (MSH). The cell line HP75, from Homo sapiens has similar characteristics.

(iii) Cells from the Pancreas

Alpha cells release glucagon and beta cells release insulin. Both cell types are involved in diabetes (Barg, 2003). Sulfonylureas are used to treat Type 2 diabetes. Sulfonylureas represent a class of drugs which bind to receptors controlling the ATP-sensitive potassium channel—the major control of voltage differences across the cellular membrane. When sulfonylureas bind to this receptor in the pancreas, they close the potassium channel thereby stimulating secretion of granules containing insulin. In the pancreas also pancreatic D and F cells secrete granules containing somatostatin and pancreatic polypeptide, respectively. Several cell lines generated from the pancreas are reported to secrete insulin: Beta-TC-6, RIN-m5F, HIT-T15, NIT-1, RIN-m, RIN-5F, TGP55. Several cell lines generated from the pancreas are reported to secrete glucagon: Beta-TC-6, RIN-m and alphaTC1 Clone 9.

(iv) Atrial Myocytes

(v) Carotid Body Glomus Cells

These release dopamine from secretory granules.

(vi) Enteroendocrine Cells

A group of cells, which may be divided into a number of populations on the basis of polypeptide hormone and biogenic amine production, found scattered throughout the gastrointestional epithelium, mainly at the base of the epithelium; their numerous small secretory granules are concentrated chiefly between the nucleus and the cell base. Their secretions affect gastrointestinal motility, pancreatic and biliary secretions, and gastrointestinal epithelial growth, as well as being regulators of other enteroendocrine products. One population, the enterochromaffin cells, secrete histamine and are involved in the regulation of acid secretion by parietal cells in the stomach and the development of gastric carcinomas (Prinz et al., 2003; Modlin and Tang, 1999).

(vii) Gastric G Cells

These release gastrin from secretory granules.

(viii) Intestinal I Cells

These release colecistokinin from secretory granules.

(ix) Kidney Juxtaglomerular Cells

These release renin from secretory granules.

(x) Pinealocytes form the Pineal Gland

Exoctine Cells

(i) Airway Submucosal Glands

Globet cells secrete mucin from granules in the airways (Rogers, 1994). Hypersecretion causes can be involved in asthma, bronchitis and cystic fibrosis (Nadel and Takeyama, 1999; Takeyama et al., 1998).

(ii) Parotid Acinar Cells/Salivary Gland Cells

These secrete amylase and other proteins from granules to make saliva (Castle et al., 2002; Castle and Castle, 1998). Impaired saliva secretion causes xerostomia or dry-mouth syndrome (Guggenheimer and Moore, 2003; Thie et al., 2002).

(iii) Gastric Chief Cells

(iv) Intestinal Globet Cells

These release mucin secretory granules.

(v) Mammary Epithelial Cells

These release casein vesicles.

(vi) Pancreatic Acinar Cells

These release zymogen granules.

Haemopoietic Cells

These include (a) eosinophils, (b) neutrophils, (c) macrophages, (d) mast cells (and cell lines derived as: MC/9), (e) basophils, (f) T-cells, and (g) platelets.

They secrete a variety of different proteins from granules (Logan et al., 2003). Lysosome related granules are secreted by (a), (e), (f) and (g). Multiple granules are released by (b), (c) and (d). Degranulation of (a-f) is related to asthma, rhinitis, eczema, food allergies and urticaria (Fireman, 2003; Miescher and Vogel, 2002). Degranulation of (e) is related to blood coagulation.

Paracrine Cells Releasing Signalling Molecules that Act Locally

(i) Myofibroblasts

These are a unique group of smooth-muscle-like fibroblasts. Through the secretion of inflammatory and anti-inflammatory cytokines, chemokines, growth factors, both lipid and gaseous inflammatory mediators, as well as extracellular matrix proteins and proteases, they play an important role in organogenesis and oncogenesis, inflammation, repair, and fibrosis in most organs and tissues.

(ii) Skin Merkel Cells

These are related to skin sensory functions and release granules by exoytosis (Tachibana and Nawa, 2002). They are the basis of a type of skin cancer, the Merkel cell carcinoma (Halata et al., 2003).

(iii) Endothelial Cells

These secrete weibel-palade body granules. Impaired secretion can lead to anomalous blood coagulation and hypertension.

(iv) Sperm

These contain acrosome granules.

(v) Eggs

These contain cortical granules.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

### REFERENCES

All the references are incorporated by reference.
Albillos, A., Dernick, G., Horstmann, H., Almers, W., Alvarez de Toledo, G., and Lindau, M. (1997). The exocytotic event in chromaffin cells revealed by patch amperometry. Nature 389, 509-512.
Ales, E., Tabares, L., Poyato, J. M., Valero, V., Lindau, M., and Alvarez de Toledo, G. (1999). High calcium concentrations shift the mode of exocytosis to the kiss-and-run mechanism. Nat Cell Biol 1, 40-44.
Anderson, K. I., and Cross, R. (2000). Contact dynamics during keratocyte motility. Curr Biol 10, 253-260.
Angleson, J. K., and Betz, W. J. (1997). Monitoring secretion in real time: capacitance, amperometry and fluorescence compared. Trends Neurosci 20, 281-287.
Ashmore, J. F., and Falk, G. (1980). Responses of rod bipolar cells in the dark-adapted retina of the dogfish, Scyliorhinus canicula. J Physiol 300, 115-150.
Avery, J., Ellis, D. J., Lang, T., Holroyd, P., Riedel, D., Henderson, R. M., Edwardson, J. M., and Jahn, R. (2000). A cell-free system for regulated exocytosis in PC12 cells. J Cell Biol 148, 317-324.
Axelrod, D. (1989). Total internal reflection fluorescence microscopy. Methods Cell Biol 30, 245-270.
Axelrod, D. (2001). Total internal reflection fluorescence microscopy in cell biology. Traffic 2, 764-774.
Bader, M. F., Holz, R. W., Kumakura, K., and Vitale, N. (2002). Exocytosis: the chromaffin cell as a model system. Ann N Y Acad Sci 971, 178-183.
Barg, S., Olofsson, C. S., Schriever-Abeln, J., Wendt, A., Gebre-Medhin, S., Renstrom, E., and Rorsman, P. (2002). Delay between fusion pore opening and peptide release from large dense-core vesicles in neuroendocrine cells. Neuron 33, 287-299.
Barg, S. (2003). Mechanisms of exocytosis in insulin-secreting B-cells and glucagon-secreting A-cells. Pharmacol Toxicol 92, 3-13.
Betz, W. J., Mao, F., and Smith, C. B. (1996). Imaging exocytosis and endocytosis. Curr Opin Neurobiol 6, 365-371.
Burgoyne, R. D., and Morgan A. Secretory granule exocytosis. Physiol Rev. 2003 83:581-632.
Burrone, J., and Lagnado, L. (1997). Electrical resonance and Ca²⁺ influx in the synaptic terminal of depolarizing bipolar cells from the goldfish retina. J Physiol 505, 571-584.
Burrone, J., and Lagnado, L. (2000). Synaptic depression and the kinetics of exocytosis in retinal bipolar cells. J Neurosci 20, 568-578.
Burrone, J., Neves, G., Gomis, A., Cooke, A., and lagnado, L. (2002). Endogenous calcium buffers regulate fast exocytosis in the synaptic terminal of retinal bipolar cells. Neuron 33, 101-112.
Carrasco, G. A., and Van de Kar, L. D. (2003). Neuroendocrine pharmacology of stress. Eur J Pharmacol 463, 235-272.
Castle, A. M., Huang, A. Y., and Castle, J. D. (2002). The minor regulated pathway, a rapid component of salivary secretion, may provide docking/fusion sites for granule exocytosis at the apical surface of acinar cells. J Cell Sci 115, 2963-2973.
Carter, T. D., Zupancic, G., Smith, S. M., Wheeler-Jones, C., and Ogden, D. (1998). Membrane capacitance changes induced by thrombin and calcium in single endothelial cells cultured from human umbilical vein. J Physiol 513 (Pt 3), 845-855.
Castle, D., and Castle, A. (1998). Intracellular transport and secretion of salivary proteins. Crit Rev Oral Biol Med 9, 4-22.
Chen, P., Hwang, T. C., and Gillis, K. D. (2001). The relationship between cAMP, Ca(2)⁺, and transport of CFTR to the plasma membrane. J Gen Physiol 118, 135-144.
Cochilla, A. J., Angleson, J. K., and Betz, W. J. (1999). Monitoring secretory membrane with FM1-43 fluorescence. Annu Rev Neurosci 22, 1-10.
Cummings DE, Schwartz MW. (2003) Genetics and pathophysiology of human obesity. Annu Rev Med. 54:453-71.
Curley K, Lawrence DS. (1999). Light-activated proteins. Curr Opin Chem Biol. 3, 84-88.
Curtis, A. S. G. (1964). The mechanism of adhesion of cells to glass. J Cell Biol 20, 199-215.
Cutolo, M., Sulli, A., Pizzorni, C., Craviotto, C., and Straub, R. H. (2003). Hypothalamic-pituitary-adrenocortical and gonadal functions in rheumatoid arthritis. Ann N Y Acad Sci 992, 107-117.
Duncan, R. R., Greaves, J., Wiegand, U. K., Matskevich, I., Bodammer, G., Apps, D. K., Shipston, M. J., and Chow, R. H. (2003). Functional and spatial segregation of secretory vesicle pools according to vesicle age. Nature 422, 176-180.
Elbashir et al. Nature 411:494 (2001).
Fireman, P. (2003). Understanding asthma pathophysiology. Allergy Asthma Proc 24, 79-83.
Gillis, K. (1995). Techniques for Membrane Capacitance Measurements. In Single Channel Recording, B. Sakmann, and E. Neher, eds. (Plenum Press), pp. 155-197.
Gingell, D., and Todd, I. (1979). Interference reflection microscopy. A quantitative theory for image interpretation and its application to cell-substratum separation measurement. Biophys J 26, 507-526.
Gomis, A., Burrone, J., and Lagnado, L. (1999). Two actions of calcium regulate the supply of releasable vesicles at the ribbon synapse of retinal bipolar cells. J Neurosci 19, 6309-6317.
Guggenheimer, J., and Moore, P. A. (2003). Xerostomia: etiology, recognition and treatment. J Am Dent Assoc 134, 61-69.
Gundelfinger, E. D., Kessels, M. M. and Qualmann, B. (2003). Temporal and spatial coordination of exocytosis and endocytosis. Nat Rev Mol Cell Biol. 4, 127-139.
Halata, Z., Grim, M., and Bauman, K. I. (2003). Friedrich Sigmund Merkel and his “Merkel cell”, morphology, development, and physiology: Review and new results. Anat Rec 271A, 225-239.
Hannon, G. J. (2002). RNA interference. Nature 418, 244-251.
Heuser, J., Zhu, Q., and Clarke, M. (1993), Proton pumps populate the contractile vacuoles of Dictyostelium amoebae. J Cell Biol 121, 1311-1327.
Holevinsky, K. O., and Nelson, D. J. (1998). Membrane capacitance changes associated with particle uptake during phagocytosis in macrophages. Biophys J 75, 2577-2586.
Holroyd, P., Lang, T., Wenzel, D., De Camilli, P., and Jahn, R. (2002). Imaging direct, dynamin-dependent recapture of fusing secretory granules on plasma membrane lawns from PC12 cells. Proc Natl Acad Sci USA 99, 16806-16811.
Horrigan, F. T., and Bookman, R. J. (1994). Releasable pools and the kinetics of exocytosis in adrenal chromaffin cells. Neuron 13, 1119-1129.
Huang, L. Y., and Neher, E. (1996). Ca(2+)-dependent exocytosis in the somata of dorsal root ganglion neurons. Neuron 17, 135-145.
Izzard, C. S., and Lochner, L. R. (1976). Cell-to-substrate contacts in living fibroblasts: an interference reflexion study with an evaluation of the technique. J Cell Sci 21, 129-159.
Kaneko, A., Pinto, L. H., and Tachibana, M. (1989). Transient calcium current of retinal bipolar cells of the mouse. J Physiol 410, 613-629.
Llobet, A., Cooke, A., and Lagnado, L. (2003). Exocytosis at the ribbon synapse of retinal bipolar cells studied in patches of presynaptic membrane. J Neuroscience 23, 2706-2714.
Logan, M. R., Odemuyiwa, S. O., and Moqbel, R. (2003). Understanding exocytosis in immune and inflammatory cells: the molecular basis of mediator secretion. J Allergy Clin Immunol 111, 923-932.
Lollike K, Lindau M. (1999). Membrane capacitance techniques to monitor granule exocytosis in neutrophils. J Immunol Methods. 232, 111-20.
Riedel D, Antonin W, Fernandez-Chacon R, Alvarez de Toledo G, Jo T, Geppert M, Valentijn J A, Valentijn K, Jamieson J D, Sudhof T C, Jahn R. (2002). Rab3D is not required for exocrine exocytosis but for maintenance of normally sized secretory granules. Mol Cell Biol. 22, 6487-6497
Mennerick, S., and Matthews, G. (1996). Ultrafast exocytosis elicited by calcium current in synaptic terminals of retinal bipolar neurons. Neuron 17, 1241-1249.
Michael, D. J., and Wightman, R. M. (1999). Electrochemical monitoring of biogenic amine neurotransmission in real time. J Pharm Biomed Anal 19, 33-46.
Miescher, S. M., and Vogel, M. (2002). Molecular aspects of allergy. Mol Aspects Med 23, 413-462.
Modlin, I. M., and Tang, L. H. (1999). Cell and tumour biology of the gastric enterochromaffin-like cell. Ital J Gastroenterol Hepatol 31 Suppl 2, S117-130.
Nadel, J. A., and Takeyama, K. (1999). Mechanisms of hypersecretion in acute asthma, proposed cause of death, and novel therapy. Pediatr Pulmonol Suppl 18, 54-55.
Neher, E., and Marty, A. (1982). Discrete changes of cell membrane capacitance observed under conditions of enhanced secretion in bovine adrenal chromaffin cells. Proc Natl Acad Sci USA 79, 6712-6716.
Neves, G., Gomis A, Lagnado L (2001). Calcium influx selects the fast mode of endocytosis in the synaptic terminal of a retinal neuron. Proceedings of the National Acadamy of Sciences-USA.
Neves, G., and Lagnado, L. (1999). The kinetics of exocytosis and endocytosis in the synaptic terminal of goldfish retinal bipolar cells. J Physiol 515, 181-202.
Niewohner, J., Weber, I., Maniak, M., Muller-Taubenberger, A., and Gerisch, G. (1997). Talin-null cells of Dictyostelium are strongly defective in adhesion to particle and substrate surfaces and slightly impaired in cytokinesis. J Cell Biol 138, 349-361.
Oshiro, T., Kakuta, Y., Maruyama, N., Fushimi, T., Okayama, H., Tamura, G., Shimura, S., and Shirato, K. (1997). Patch-clamp characterization of secretory process in human basophils. Int Arch Allergy Immunol 112, 336-340.
Parmer, R. J., and Zinder, O. (2002). Catecholaminergic pathways, chromaffin cells, and human disease. Ann N Y Acad Sci 971, 497-505.
Parson T D, Coorsen J R, Horstmann H, Almers W (1995) Docked granules, the exocytic burst and the need for ATP hydrolysis in endocrine cells. Neuron 15:1085-1096.
Parsons, T. D., Lenzi, D., Almers, W., and Roberts, W. M. (1994). Calcium-triggered exocytosis and endocytosis in an isolated presynaptic cell: capacitance measurements in saccular hair cells. Neuron 13, 875-883.
Prinz, C., Zanner, R., and Gratzl, M. (2003). Physiology of gastric enterochromaffin-like cells. Annu Rev Physiol 65, 371-382.
Ploem, J. S. (1975). Refelection-contrast microscopy as a tool for investigating the attachment of living cells to a glass surface. In Mononuclear Phagocytes in Immunity, Infection and Pathology, R. Van Furth, ed. (London, Blackwell Scientific Publications), pp. 405-421.
Pyle, J. L., Kavalali, E. T., Piedras-Renteria, E. S., and Tsien, R. W. (2000). Rapid reuse of readily releasable pool vesicles at hippocampal synapses. Neuron 28, 221-231.
Rahamimoff, R., and Fernandez, J. M. (1997). Pre- and postfusion regulation of transmitter release. Neuron 18, 17-27.
Rettig, J., and Neher, E. (2002). Emerging roles of presynaptic proteins in Ca++−triggered exocytosis. Science 298, 781-785.
Rogers, D. F. (1994). Airway goblet cells: responsive and adaptable front-line defenders. Eur Respir J 7, 1690-1706.
Royle, S. R. and Murrell-Lagnado, R. D. (2003). Constitutive cycling: a general mechanism to regulate cell surface proteins. BioEssays 25, 39-46.
Ryan, T. A. (2001). Presynaptic imaging techniques. Curr Opinion Neurobiol. 11, 544-549.
Schiavo, G., Matteoli, M., and Montecucco, C. (2000). Neurotoxins affecting neuroexocytosis. Physiol Rev 80, 717-766.
Senda, T. (1995). Mechanisms of secretory granule transport and exocytosis in anterior pituitary cells. Ital J Anat Embryol 100 Suppl 1, 219-229.
Seward, E. P., and Nowycky, M. C. (1996). Kinetics of stimulus-coupled secretion in dialyzed bovine chromaffin cells in response to trains of depolarizing pulses. J Neurosci 16, 553-562.
Sikdar, S. K., Kreft, M., and Zorec, R. (1998). Modulation of the unitary exocytic event amplitude by cAMP in rat melanotrophs. J Physiol 511 (Pt 3), 851-859.
Stevens, C. F., and Williams, J. H. (2000). “Kiss and run” exocytosis at hippocampal synapses. Proc Natl Acad Sci USA 97, 12828-12833.
Steyer, J. A., Horstmann, H., and Almers, W. (1997). Transport, docking and exocytosis of single secretory granules in live chromaffin cells. Nature 388, 474-478.
Sun, J. Y., and Wu, L. G. (2001). Fast kinetics of exocytosis revealed by simultaneous measurements of presynaptic capacitance and postsynaptic currents at a central synapse. Neuron 30, 171-182.
Tachibana, T., and Nawa, T. (2002). Recent progress in studies on Merkel cell biology. Anat Sci Int 77, 26-33.
Takei, T., Yasufuku-Takano, J., Takano, K., Fujita, T., and Yamashita, N. (1998). Effect of Ca2+ and cAMP on capacitance-measured hormone secretion in human GH-secreting adenoma cells. Am J Physiol 275, E649-654.
Takeyama, K., Agusti, C., Ueki, I., Lausier, J., Cardell, L. O., and Nadel, J. A. (1998). Neutrophil-dependent goblet cell degranulation: role of membrane-bound elastase and adhesion molecules. Am J Physiol 275, L294-302.
Taraska, J. W., Perrais, D., Ohara-Imaizumi, M., Nagamatsu, S., and Almers, W. (2003). Secretory granules are recaptured largely intact after stimulated exocytosis in cultured endocrine cells. Proc Natl Acad Sci USA 100, 2070-2075.
Thie, N. M., Kato, T., Bader, G., Montplaisir, J. Y., and Lavigne, G. J. (2002). The significance of saliva during sleep and the relevance of oromotor movements. Sleep Med Rev 6, 213-227.
Thomas, P., and Waring, D. W. (1997). Modulation of stimulus-secretion coupling in single rat gonadotrophs. J Physiol 504 (Pt 3), 705-719.
Vaidyanathan, V. V., Yoshino, K., Jahnz, M., Dorries, C., Bade, S., Nauenburg, S., Niemann, H., and Binz, T. (1999). Proteolysis of SNAP-25 isoforms by botulinum neurotoxin types A, C, and E: domains and amino acid residues controlling the formation of enzyme-substrate complexes and cleavage. J Neurochem 72, 327-337.
Valtorta, F., Meldolesi, J., and Fesce, R. (2001). Synaptic vesicles: is kissing a matter of competence? Trends Cell Biol 11, 324-328.
Verschueren, H. (1985). Interference reflection microscopy in cell biology: methodology and applications. J Cell Sci 75, 279-301.
von Gersdorff, H., and Matthews, G. (1994). Dynamics of synaptic vesicle fusion and membrane retrieval in synaptic terminals. Nature 367, 735-739.
Weber, I. (2003). Reflection interference contrast microscopy. Methods Enzymol 361, 34-47.
Zenisek, D., Davila, V., Wan, L., and Almers, W. (2003). Imaging calcium entry sites and ribbon structures in two presynaptic cells. J Neurosci 23, 2538-2548.
Zenisek, D., Steyer, J. A., and Almers, W. (2000). Transport, capture and exocytosis of single synaptic vesicles at active zones. Nature 406, 849-854.
Zenisek, D., Steyer, J. A., Feldman, M. E., and Almers, W. (2002). A membrane marker leaves synaptic vesicles in milliseconds after exocytosis in retinal bipolar cells. Neuron 35, 1085-1097.

1. A method of assaying exocytosis or endocytosis, comprising:
a. providing a transparent substrate with at least one cell membrane mounted thereon apposing the substrate,
b. provoking a change in an amount of exocytosis or endocytosis at the membrane,
c. obtaining light signals from a region of the substrate which the membrane apposes using interference reflection microscopy, and
d. analysing the light signals to detect the exocytosis or endocytosis at the membrane.

2. A method of assaying exocytosis or endocytosis, comprising:
a. providing a transparent substrate with at least one cell membrane mounted thereon apposing the substrate,
b. obtaining first light signals from a region of the substrate which the membrane apposes using interference reflection microscopy
c. provoking a change in an amount of exocytosis or endocytosis at the membrane,
d. obtaining second light signals from said region of the substrate using interference reflection microscopy, and
e. analysing the first and second light signals to detect the change in the amount of exocytosis or endocytosis at the membrane.

3. A method according to claim 1 or 2, wherein the change in the amount of exocytosis or endocytosis is provoked by stimulating exocytosis or endocytosis at the membrane.

4. A method according to claim 1 or 2, wherein the change in the amount of exocytosis or endocytosis is provoked by inhibiting exocytosis or endocytosis at the membrane.

5. A method of monitoring exocytosis or endocytosis mediated by organelles of less 500 nm diameter, comprising:
a. providing a transparent substrate with at least one cell membrane mounted thereon apposing the substrate,
b. obtaining light signals from a region of the substrate which the membrane apposes using interference reflection microscopy, and
c. analysing the light signals to detect exocytosis or endocytosis at the membrane mediated by said organelles.

6. A method according to any one of claims 1, 2 or 5, wherein a plurality of cell membranes are mounted on the substrate and light signals are obtained simultaneously therefrom.

7. A method according to any one of claims 1, 2 or 5, wherein the transparent substrate is provided with at least one cell mounted thereon, a portion of the cell surface membrane apposing the substrate.

8. A method for screening a population of cells to identify cells with an altered phenotype with respect to exocytosis or endocytosis, wherein the method comprises:
a. for each cell of the population:
(1) providing a transparent substrate with the cell mounted thereon such that a portion of its surface membrane apposes the substrate,
(2) obtaining light signals from a region of the substrate which the membrane apposes using interference reflection microscopy, and
(3) analysing the light signals to determine an amount of exocytosis or endocytosis at the membrane; and
b. comparing the amounts of exocytosis or endocytosis thus-determined to identify those cells of the population which have an altered phenotype with respect to exocytosis or endocytosis.

9. A method according to any one of claims 1, 2, 5 or 8, wherein the obtained light signals are in the form of images of said region of the substrate.

10. A method according to any one of claims 1, 2, 5 or 8, wherein the analysing step comprises identifying individual cell organelles which mediate exocytosis or endocytosis.

11. A method according to any one of claims 1, 2, 5 or 8, wherein the analysing step comprises measuring a total amount of light reflected from said region of the substrate.

12. A method according to any one of claims 1, 2, 5 or 8, wherein the cell membrane is from a cell selected from the group consisting of: neurons, endocrine cells, exocrine cells, paracrine cells, haemopoetic cells, epitheleial cells, endothelial cells, melanocytes, sperm and eggs.

13. A method according to any one of claims 1, 2, 5 or 8, wherein the cell membrane is from a cell selected from the group consisting of: endocrine cells, exocrine cells, paracrine cells, haemopoetic cells, epitheleial cells, endothelial cells, melanocytes, sperm and eggs.

14. A method according to any one of claims 1, 2, 5 or 8, wherein the exocytosis or endocytosis is mediated by vesicles.

15. A method according to any one of claims 1, 2, 5 or 8, wherein the exocytosis or endocytosis is mediated by secretory granules.

16. An apparatus for monitoring, assaying or screening exocytosis or endocytosis comprising:
a. a transparent substrate on which at least one cell membrane can be mounted to appose the substrate,
b. an interference reflection microscope system configured to obtain light signals from the membrane, and
c. a computerised signal analyser operatively connected to the microscope system, the signal analyser being adapted to detect exocytosis or endocytosis at the membrane in the light signals.

17. An apparatus according to claim 16, wherein the substrate forms part of a multi-well plate.

18. An apparatus according to claim 17, further comprising a first transport device for moving the multi-well plate relative to the microscope system such that light signals can be obtained from successive wells of the multi-well plate.

19. An apparatus according to claim 17, wherein the microscope system has a plurality of objective lenses to allow simultaneous light signal collection from respective wells of the multi-well plate.
